(19) United States
(12) Patent Application Publication
(10) Pub. No.: US 2026/0250774 A1
(43) Pub. Date: Aug. 27, 2026

(54) ULTRA-SENSITIVE TARGET NUCLEIC ACID ENRICHMENT DETECTION METHOD BASED ON PROGRAMMABLE NUCLEASE

(71) Applicants: INSTITUTE OF MEDICINE#BASIC SCIENCES AND CANCER, CHINESE ACADEMY OF SCIENCES(PREPARATORY), Hangzhou (CN); HANGZHOU EZDX TECHNOLOGY CO., LTD., Hangzhou (CN)

(72) Inventors: Jinzhao SONG, Hangzhou (CN); Yue SHEN, Hangzhou (CN); Jieer YING, Hangzhou (CN)

(73) Assignees: INSTITUTE OF MEDICINE#BASIC SCIENCES AND CANCER, CHINESE ACADEMY OF SCIENCES(PREPARATORY), Hangzhou (CN); HANGZHOU EZDX TECHNOLOGY CO., LTD., Hangzhou (CN)

(21) Appl. No.: 19/158,055

(22) PCT Filed: Jun. 21, 2023
(86) PCT No.: PCT/CN2023/101662

§ 371 Date: Aug. 20, 2025

(30) Foreign Application Priority Data
Feb. 20, 2023 (CN) 202310133037.0

(51) Int. Cl. C12Q 1/6886; C12N 9/22; C12N 15/11; C12Q 1/6806

(52) U.S. Cl. CPC C12Q 1/6886; C12N 9/226; C12N 15/11; C12Q 1/6806; C12N 2310/20; C12Q 2600/154; C12Q 2600/156

(57) ABSTRACT

Provided is a programmable nuclease based ultra-sensitive method for enriching and detecting a target nucleic acid. Based on specific cleavage of a programmable nuclease, by designing and optimizing an sgRNA, a guide DNA or a guide RNA, the enrichment method uses the sgRNA, the guide DNA, or the guide RNA, and a Cas protein or an Ago protein to form a ribonucleoprotein complex. Thus, non-target nucleic acids can be removed continuously and specifically while amplifying at a constant temperature. The number of target nucleic acids in a sample continuously increases exponentially, and the target nucleic acids such as low-abundance mutant genes and methylated DNAs are enriched or detected.

### CROSS REFERENCE TO THE RELATED APPLICATIONS

This application is the national phase entry of International Application No. PCT/CN2023/101662, filed on Jun. 21, 2023, which is based upon and claims priority to Chinese Patent Application No. 202310133037.0, filed on Feb. 20, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBLZXC022_Sequence_Listing.xml, created on 09/19/2025, and is 40,812 bytes in size.

### TECHNICAL FIELD

The present disclosure relates to the technical field of biology, and in particular to a programmable nuclease based ultra-sensitive method for enriching and detecting a target nucleic acid.

### BACKGROUND

Somatic mutations are closely related to tumorigenesis, and mutation detection is of great significance for early disease screening, precise treatment decision, and recurrence monitoring. In recent years, non-invasive diagnosis represented by a liquid biopsy has become a trend. The liquid biopsy is to analyze a tumor genotype by detecting free nucleic acid fragments from a tumor in a body fluid, and thus is superior in non-invasion, dynamic monitoring proneness, etc. However, the liquid biopsy is confronted by the following problems: 1) Since mutant nucleic acids have high sequence homology to wild-type nucleic acids with the mere difference of a single nucleotide or a few nucleotides, challenges are brought to identification of low-abundance mutant alleles from a large number of free wild-type alleles. 2) Detection of low-abundance methylated genes in a body fluid is also confronted by the similar problem because the methylated genes and non-methylated genes have the same sequence and merely differ in base modifications. In consequence, challenges are posed for identifying the low-abundance methylated genes from a large number of free non-methylated genes.

At present, for high-sensitive detection of mutant alleles, it is common practice to detect weak signals through next-generation sequencing (NGS) or brute force counting of a digital polymerase chain reaction (PCR). Especially in detection of minimal residual disease (MRD), ultra-low-abundance mutant genes are detected mainly based on ultra-deep (50000×) NGS sequencing that has a high cost, long time consumption, and difficulties in popularizing to clinical practice. Thus, it is a pressing issue to develop a low-cost ultra-sensitive method for detecting an ultra-low-abundance mutant allele, to facilitate clinical application.

In recent years, the clustered regularly interspaced short palindromic repeats (CRISPR)-CRISPR-associated (CRISPR-Cas) programmable nuclease based diagnostic technology has become a hot research topic. A CRISPR-Cas system is an adaptive immune defense system formed by archaebacteria and most bacteria during biological evolution to resist virus invasion. It consists of a Cas effector protein and a guide ribonucleic acid (RNA). Under the guidance of the guide RNA, the Cas protein recognizes and cleaves a target sequence including a protospacer adjacent motif (PAM) site at 5′end. According to a CRISPR-Cas based method for detecting a low-abundance mutant allele, the Cas protein specifically cleaves a wild-type allele through special guide RNA design. Thus, a large number of wild-type nucleic acids are removed and mutant alleles are retained, to improve the sensitivity of downstream analysis. Moreover, some researchers have established a similar method for detecting a low-abundance mutant allele by forming complexes by argonaute proteins (Ago) and guide deoxyribonucleic acids (DNA).

Cleavage of the Cas protein is limited by the PAM site. When mutations result in disruption of the PAM site, the Cas protein conducts target cleavage on a large number of wild-type nucleic acids in the same sample, with mutant nucleic acids retained. For mutations at the PAM site, some researchers have developed methods for detecting low-abundance mutant alleles, such as depletion of abundant sequences by hybridization (DASH) and CRISPR-mediated, ultra-sensitive detection of target DNA using PCR (CUT-PCR). The argonaute protein is not limited by the PAM site. Some researchers have developed a method for detecting a low-abundance mutant allele, such as nucleic acid enrichment via DNA guided argonaute from Thermus thermophilus (NAVIGATER).

Although the DASH method and the CUT-PCR method improve the sensitivity of downstream analysis to some extent, the effectiveness of existing programmable endonuclease based detection methods is affected by invalid binding events between enzyme and target. Due to the ultra-slow dissociation efficiency of cas9, some wild-type allele targets are protected from cleavage, and non-specific off-target cleavage depletes the extremely-rare mutant alleles. The NAVIGATER method has the similar problem. To overcome these shortcomings, the researchers employ multiple rounds of selective cleavage of wild-type alleles, followed by the polymerase chain reaction, to enrich fragments of the mutant alleles 10-fold. Despite these improvements, the high sensitivity of mutant allele detection still requires next-generation sequencing (NGS) or relies on the digital PCR, making these methods laborious and expensive.

For detection of low-abundance methylated genes during the liquid biopsy, the above technologies with similar problems have difficulties in satisfying requirements.

### SUMMARY

An objective of the present disclosure is to provide a programmable nuclease based ultra-sensitive method for enriching tumor-related genes. The method of the present disclosure can efficiently enrich low-abundance mutant genes and methylated genes, and can be automated through a microfluidic chip technology.

To achieve the above objective, the following technical solution is employed.

A ribonucleoprotein complex is provided, which includes:
a component (a), which includes a nucleic acid region capable of complementarily matching a target nucleic acid and 0 bp-4 bp mismatched fragments; or a nucleic acid region capable of complementarily matching a target nucleic acid, where an adjacent protospacer adjacent motif (PAM) includes a mutation-susceptible site;
a component (b), which is capable of binding with the target nucleic acid and breaking a strand of the target nucleic acid;
the component (a) is selected from a trans-activating clustered regularly interspaced short palindromic repeats ribonucleic acid (tracrRNA), and/or a clustered regularly interspaced short palindromic repeats ribonucleic acid (crRNA), and/or a single guide RNA (sgRNA), and/or a derivative of the tracrRNA, and/or a derivative of the crRNA, and/or a derivative of the sgRNA, and/or a guide deoxyribonucleic acid (DNA), and/or a guide RNA; the component (b) includes a clustered regularly interspaced short palindromic repeats (CRISPR)-associated (Cas) protein, and/or a derivative of the Cas protein, and/or an argonaute (Ago) protein, and/or a derivative of the Ago protein; and the component (a) and the component (b) are capable of binding with each other. Preferably, the component (a) may carry a marker, where the marker includes streptavidin or biotin.

Preferably, a length of a mismatched nucleotide fragment is 0 bp, 1 bp, 2 bp, 3 bp, or 4 bp.

Preferably, the component (a) carries a chemical modification, where the chemical modification includes a sulfur-substituted oxygen or methoxy modification.

Preferably, the sgRNA, the guide DNA, or the guide RNA in the component (a) includes the mismatched nucleotide fragment. Through the mismatched nucleotide fragment designed by the present disclosure, an application range of the sgRNA, the guide DNA, or the guide RNA can be expanded, and more mutations can be recognized by the sgRNA, the guide DNA, or the guide RNA.

Preferably, the PAM adjacent to the sgRNA in the component (a) includes a hot tumor mutation site.

Preferably, a length of a spacer sequence of the sgRNA in/of the component (a) is 16 nt-22 nt.

More preferably, sequences of the sgRNA are as follows:

(SEQ ID NO: 1)
UCUUAAUUCCUUGAUAGCGA;
(SEQ ID NO: 2)
UAGCUACAGUGAACUCUCGA;
(SEQ ID NO: 3)
UAGCUACAGUGAAAUCACGA;
(SEQ ID NO: 4)
GCUACAGUGAACUCUCGA;
(SEQ ID NO: 5)
GUCUAGCUACAGUGAAA;
(SEQ ID NO: 6)
GUCUAGCUGCAGUGAAA;
(SEQ ID NO: 27)
GGCAGCCGAAGGGCAUGAGC;
(SEQ ID NO: 28)
GGCAGCCGAAGAGCAUGAGC;
(SEQ ID NO: 37)
AAUUUUUGUUUGAGUGGUUG;
(SEQ ID NO: 42)
UCCAGCUGUAUCCAGUAUGU.

More preferably, sequences of the guide DNA are as follows:

forward guide:
(SEQ ID NO: 43)
p-TAGATTTCACTGTAGC-3';
and
reverse guide:
(SEQ ID NO: 44)
p-TTCTAGCTACAGTGAA-3'.

Preferably, the component (b) includes at least one of the following: Cas12a, SacCas9, CjCas9, SpCas9, NmCas9, Sp-Cas9 HF1, evoCas9, HypaCas9, HiFi Cas9, Sniper-Cas9, xCas9, eSpCas9 1.1, SuperFi Cas9, SaCas9, SaCas9-IF, efSaCas9, ScCas9, Cas9-Sc++, Cas9 HiFi-Sc++, SpaCas9, SpaCas9-IF, FnCas9, AnaCas9, SpyCas9, FnCas12a, LbCas12a, AsCas12a, CbAgo, TtAgo, PfAgo, KmAgo, or KpAgo.

More preferably, the Cas12a is from at least one of the following species: Francisella novicida; Acidaminococcus; or Lachnospiraceae.

More preferably, the SacCas9 is from the species Staphylococcus aureus.

More preferably, the CjCas9 is from the species Campylobacter jejuni.

More preferably, the SpCas9 is from the species Streptococcus pyogenes.

More preferably, the NmCas9 is from the species Neisseria meningitidis.

More preferably, the SaCas9 is from the species Staphylococcus aureus.

More preferably, the ScCas9 is from the species Streptococcus canis.

More preferably, the SpaCas9 is from the species Streptococcus pasteurianus.

Preferably, a final concentration of the component (a) in the ribonucleoprotein complex is 0.1 μM-2 μM.

Preferably, a final concentration of the component (b) in the ribonucleoprotein complex is 0.1 μM-3 μM.

Preferably, the ribonucleoprotein complex further includes a hydrogen ion buffer.

More preferably, a final concentration of the hydrogen ion buffer in the ribonucleoprotein complex is 1 mM-2 M.

Use of the above ribonucleoprotein in enriching and/or detecting mutant genes is further disclosed in the present disclosure.

Use of the above ribonucleoprotein in enriching and/or detecting methylated DNAs is further disclosed in the present disclosure.

Preferably, the gene includes at least one of the following: EGFR, BRAF, PIK3CA, TP53, LRP1B, APC, CYP1A1, NPO1, EPHX1, KRAS, BRCA1, BRCA2, MET, MLH1, MSH2, MSH3, MSH6, PALB2, BMPR1A, SMAD4, STK11, PTEN, AXIN2, BLM, BUB1B, CDH1, CEP57, CHEK2, ENG, EPCAM, FLCN, GALNTI2, GREM1, FAT4, KMT2D, KMT2C, ARID1A, FAT1, PTEN, ATM, ZFHX3, CREBBP, GRIN2A, NRAS, or NF1.

A method for preparing a ribonucleoprotein complex is further disclosed in the present disclosure. The method includes: mixing and incubating Cas9, an sgRNA, and a hydrogen ion buffer; and mixing and incubating Ago, a guide DNA or RNA, and a hydrogen ion buffer.

Preferably, the hydrogen ion buffer includes a hydroxyethylpiperazine ethane sulfonic acid (HEPES).

Preferably, in preparation of the ribonucleoprotein complex, a final concentration of the Cas9 used is 0.1 μM-3 μM.

More preferably, in the preparation of the ribonucleoprotein complex, the final concentration of the Cas9 used is 2.5 μM.

Preferably, in the preparation of the ribonucleoprotein complex, a final concentration of the sgRNA used is 0.1 μM-2 μM.

More preferably, in the preparation of the ribonucleoprotein complex, the final concentration of the sgRNA used is 2.5 μM.

Preferably, in the preparation of the ribonucleoprotein complex, a final concentration of the HEPES used is 1 mM-2 M.

Preferably, in the preparation of the ribonucleoprotein complex, the final concentration of the HEPES used is 1 M.

Preferably, in the preparation of the ribonucleoprotein complex, a reaction temperature is 34° C.-39° C., and a reaction time is 3 min-18 min.

More preferably, in the preparation of the ribonucleoprotein complex, the reaction temperature is 37° C., and the reaction time is 10 min.

A reaction system for enriching mutant genes of a programmable enzyme and/or enriching methylated DNAs is further disclosed in the present disclosure. The reaction system includes the above ribonucleoprotein complex.

Preferably, the ribonucleoprotein complex includes:
a component (a), which includes a nucleic acid region capable of complementarily matching a target nucleic acid and 0 bp-4 bp mismatched fragments; and
a component (b), which is capable of binding with the target nucleic acid and breaking a strand of the target nucleic acid;
where the component (a) is selected from a tracrRNA, and/or a crRNA, and/or an sgRNA, and/or a derivative of the tracrRNA, and/or a derivative of the crRNA, and/or a derivative of the sgRNA, and/or a guide DNA, and/or a guide RNA; the component (b) includes a Cas protein, and/or a derivative of the Cas protein, and/or an Ago protein, and/or a derivative of the Ago protein; and the component (a) and the component (b) are capable of binding with each other. Preferably, the component (a) carries a marker, where the marker includes streptavidin or biotin.

Preferably, the length of the mismatched nucleotide fragment is 0 bp, 1 bp, 2 bp, 3 bp, or 4 bp.

More preferably, the component (a) carries a chemical modification, where the chemical modification includes a sulfur-substituted oxygen or methoxy modification.

More preferably, the sgRNA, the guide DNA, or the guide RNA in the component (a) includes the mismatched nucleotide fragment.

More preferably, a length of a spacer sequence of the sgRNA, the guide DNA, or the guide RNA in the component (a) is 16 nt-22 nt.

Furthermore preferably, sequences of the sgRNA are as follows:

(SEQ ID NO: 1)
UCUUAAUUCCUUGAUAGCGA;
(SEQ ID NO: 2)
UAGCUACAGUGAACUCUCGA;
(SEQ ID NO: 3)
UAGCUACAGUGAAAUCACGA;
(SEQ ID NO: 4)
GCUACAGUGAACUCUCGA;
(SEQ ID NO: 5)
GUCUAGCUACAGUGAAA;
(SEQ ID NO: 6)
GUCUAGCUGCAGUGAAA;
(SEQ ID NO: 27)
GGCAGCCGAAGGGCAUGAGC;
(SEQ ID NO: 28)
GGCAGCCGAAGAGCAUGAGC;
(SEQ ID NO: 37)
AAUUUUUGUUUGAGUGGUUG;
(SEQ ID NO: 42)
UCCAGCUGUAUCCAGUAUGU.

More preferably, sequences of the guide DNA are as follows:

forward guide:
(SEQ ID NO: 43)
p-TAGATTTCACTGTAGC-3';
and
reverse guide:
(SEQ ID NO: 44)
p-TTCTAGCTACAGTGAA-3'.

Preferably, the component (b) includes at least one of the following: Cas12a, SacCas9, CjCas9, SpCas9, NmCas9, Sp-Cas9 HF1, evoCas9, HypaCas9, HiFi Cas9, Sniper-Cas9, xCas9, eSpCas9 1.1, SuperFi Cas9, SaCas9, SaCas9-HF, efSaCas9, ScCas9, Cas9-Sc++, Cas9 HiFi-Sc++, SpaCas9, SpaCas9-HF, FnCas9, AnaCas9, SpyCas9, FnCas12a, LbCas12a, AsCas12a, CbAgo, TtAgo, PfAgo, KmAgo, or KpAgo.

Furthermore preferably, the Cas12a is from at least one of the following species: Francisella novicida; Acidaminococcus; or Lachnospiraceae.

Furthermore preferably, the SacCas9 is from the species Staphylococcus aureus.

Furthermore preferably, the CjCas9 is from the species Campylobacter jejuni.

Furthermore preferably, the SpCas9 is from the species Streptococcus pyogenes.

Furthermore preferably, the NmCas9 is from the species Neisseria meningitidis.

More preferably, the SaCas9 is from the species Staphylococcus aureus.

More preferably, the ScCas9 is from the species Streptococcus canis.

More preferably, the SpaCas9 is from the species Streptococcus pasteurianus.

More preferably, a final concentration of the component (a) in the ribonucleoprotein complex is 0.1 μM-2 μM.

More preferably, a final concentration of the component (b) in the ribonucleoprotein complex is 0.1 μM-3 μM.

More preferably, the ribonucleoprotein complex further includes a hydrogen ion buffer.

Furthermore preferably, a final concentration of the hydrogen ion buffer in the ribonucleoprotein complex is 1 mM-2 M.

Preferably, a final concentration of the ribonucleoprotein complex in the reaction system is 0.1 μM-2 μM.

Preferably, the reaction system further includes a primer, a gene of interest, an enzyme, and an auxochrome.

Preferably, the reaction system further includes deoxy-ribonucleoside triphosphate (dNTP) and a single-stranded binding protein.

More preferably, the gene of interest includes a genomic DNA or a cell-free DNA.

Furthermore preferably, the gene of interest includes the genomic DNA extracted from at least one of the following cell lines:
an EGFR 19del wild-type cell line; an EGFR 19 E746_A750 del (2235-2249del) cell line; an EGFR 19 E746_A750 del (2236-2250del) cell line; a BRAF V600E mutant cell line; a BRAF V600E wild-type cell line; or a B-CPAP cell line.

More preferably, the primer includes a nucleotide sequence capable of binding with the gene of interest and guiding synthesis.

Furthermore preferably, the primer includes at least one of the following sequences:

(SEQ ID NO: 15)
GCATGTGGCACCATCTCACA;
(SEQ ID NO: 16)
AGAGCAGCTGCCAGACATGA;
(SEQ ID NO: 19)
CTACACCTCAGATATATTTC;
(SEQ ID NO: 20)
TGGATCCAGACAACTGT;
(SEQ ID NO: 23)
TACGTGATGGCCAGCGTGGA;
(SEQ ID NO: 24)
ACTGGGAGCCAATATTGT;
(SEQ ID NO: 33)
TCGTTAAATAGATACGTTACGC;
(SEQ ID NO: 34)
TAAAAACTAAAAACTTTCCGCG;
(SEQ ID NO: 35)
TCGTTAAATAGATACGTTACGC;
(SEQ ID NO: 36)
CAACGCCTCGAAACCTACG;
(SEQ ID NO: 38)
CCCCCAGGATTCTTACAGAAAACAAGTGGT;
(SEQ ID NO: 39)
GCAAATACACAGAGGAAGCCTTCGCCTGTCCTC;
(SEQ ID NO: 40)
CAAGTGGTTATAGATGGTGA;
(SEQ ID NO: 41)
CGCCTGTCCTCATGTATTGG.

More preferably, the enzyme includes a DNA polymerase and/or a recombinase.

More preferably, the auxochrome includes MgOAc.

Preferably, the above reaction system may be configured to enrich mutant alleles having a minor allele frequency (MAF)≥0.01%.

Use of the above reaction system is further disclosed in the present disclosure. The use includes at least one of the following:
(1) target-binding a target nucleic acid fragment;
(2) target-cleaving a target nucleic acid fragment;
(3) enriching mutant genes;
(4) detecting mutant genes;
(5) enriching methylated DNAs; or
(6) detecting methylated DNAs.

Preferably, the above enriching includes automated enriching; and the above detecting includes automated detecting.

More preferably, the automated enriching includes enriching through a microfluidic chip; and the automated detecting includes detecting through a microfluidic chip.

Furthermore preferably, the microfluidic chip is divided into three parts and consists of a top package sheet, a bottom package sheet, and an intermediate reaction layer.

Furthermore preferably, a structure of the top package sheet includes a microfluidic chip mounting hole; a microfluidic chip package positioning hole; and a microfluidic chip sample feeding hole.

Furthermore preferably, a structure of the intermediate reaction layer includes a microfluidic chip package positioning hole; a microfluidic chip mounting hole; a pre-amplification reaction chamber; a siphon valve; a digestion reaction chamber; a pre-distribution chamber; a polymerase chain reaction (PCR) chamber; a waste liquid chamber; a capillary valve; and a gas passage.

Furthermore preferably, a structure of the bottom package sheet includes a microfluidic chip mounting hole; a microfluidic chip package positioning hole; a ribonuclease A (RNase A) adding chamber; and a proteinase K adding chamber.

Preferably, the mutant gene includes a cancer-related gene.

Preferably, the cancer-related gene includes at least one of the following:
EGFR, BRAF, PIK3CA, TP53, LRP1B, APC, CYP1A1, NP01, EPHX1, KRAS, BRCA1, BRCA2, MET, MLH1, MSH2, MSH3, MSH6, PALB2, BMPR1A, SMAD4, STK11, PTEN, AXIN2, BLM, BUB1B, CDH1, CEP57, CHEK2, ENG, EPCAM, FLCN, GALNTI2, GREM1, FAT4, KMT2D, KMT2C, ARID1A, FAT1, PTEN, ATM, ZFHX3, CREBBP, GRIN2A, NRAS, or NF1.

Preferably, the methylated DNA includes a cancer-related methylated gene and/or a promoter of the cancer-related methylated gene.

Preferably, the cancer-related methylated gene includes at least one of the following:
PCDH-10, BRCA1, RASSF1A, ESR1, APC, p14ARF, p161NK4a, DAPK, CDH1, RUNX3, TFPI2, SFRP5, HIC1, PAX5, PGR, THBS1, ESR, COL23A1, C2CD4D, WNT6, OPCML, ZNF154, RARb2, ATM, MGMT, GSTP1, MIR129-2, LINC01158, CCDC181, PRKCB, TBR1, ZNF781, MARCH11, VWC2, SLC9A3, HOXA7, Septin9, IKZF1, BCAT1, hMLH1, WIF1, CDKN2A, SHOX2, 3OST2, ASSF1A, RARb, PITX2, NID2, NEUROG2, or HOXA1;

The promoter includes at least one of promoters of the following genes:
PCDH-10, BRCA1, RASSF1A, ESR1, APC, p14ARF, p16INK4a, DAPK, CDH1, RUNX3, TFPI2, SFRP5, HIC1, PAX5, PGR, THBS1, ESR, COL23A1, C2CD4D, WNT6, OPCML, ZNF154, RARb2, ATM, MGMT, GSTP1, MIR129-2, LINC01158, CCDC181, PRKCB, TBR1, ZNF781, MARCH11, VWC2, SLC9A3, HOXA7, Septin9, IKZF1, BCAT1, hMLH1, WIF1, CDKN2A, SHOX2, 3OST2, ASSF1A, RARb, PITX2, NID2, NEUROG2, or HOXA1.

Use of the above ribonucleoprotein complex is further disclosed in the present disclosure.

The use includes at least one of the following:
(1) enriching gene mutation;
(2) detecting gene mutation;
(3) enriching methylated DNAs; or
(4) detecting methylated DNAs.

More preferably, the gene includes: EGFR, BRAF, PIK3CA, TP53, LRP1B, APC, CYP1A1, NP01, EPHX1, KRAS, BRCA1, BRCA2, MET, MLH1, MSH2, MSH3, MSH6, PALB2, BMPR1A, SMAD4, STK11, PTEN, AXIN2, BLM, BUB1B, CDH1, CEP57, CHEK2, ENG, EPCAM, FLCN, GALNTI2, GREM1, FAT4, KMT2D, KMT2C, ARID1A, FAT1, PTEN, ATM, ZFHX3, CREBBP, GRIN2A, NRAS, or NF1.

Use of a Cas protein is further disclosed in the present disclosure. The use includes at least one of the following:
(1) enriching gene mutation;
(2) detecting gene mutation;
(3) enriching methylated DNAs; or
(4) detecting methylated DNAs.

Use of an Ago protein is further disclosed in the present disclosure. The use includes at least one of the following:
(1) enriching gene mutation;
(2) detecting gene mutation;
(3) enriching methylated DNAs; or
(4) detecting methylated DNAs.

Preferably, the gene includes at least one of the following: EGFR, BRAF, PIK3CA, TP53, LRP1B, APC, CYP1A1, NPO1, EPHX1, KRAS, BRCA1, BRCA2, MET, MLH1, MSH2, MSH3, MSH6, PALB2, BMPR1A, SMAD4, STK11, PTEN, AXIN2, BLM, BUB1B, CDH1, CEP57, CHEK2, ENG, EPCAM, FLCN, GALNTI2, GREM1, FAT4, KMT2D, KMT2C, ARID1A, FAT1, PTEN, ATM, ZFHX3, CREBBP, GRIN2A, NRAS, or NF1.

Preferably, the Cas protein includes at least one of the following: Cas12a, SacCas9, CjCas9, SpCas9, NmCas9, Sp-Cas9 HF1, evoCas9, HypaCas9, HiFi Cas9, Sniper-Cas9, xCas9, eSpCas9 1.1, SuperFi Cas9, SaCas9, SaCas9-IF, efSaCas9, ScCas9, Cas9-Sc++, Cas9 HiFi-Sc++, SpaCas9, SpaCas9-IF, FnCas9, AnaCas9, SpyCas9, FnCas12a, LbCas12a, or AsCas12a.

Preferably, the Ago protein includes at least one of the following: CbAgo, TtAgo, PfAgo, KmAgo, or KpAgo.

A kit for enriching mutant genes of a programmable enzyme is further disclosed in the present disclosure. The kit includes the above ribonucleoprotein complex.

Preferably, the ribonucleoprotein complex includes:
a component (a), which includes a nucleic acid region capable of complementarily matching a target nucleic acid and 0 bp-4 bp mismatched fragments; and
a component (b), which is capable of binding with the target nucleic acid and breaking a strand of the target nucleic acid;
where the component (a) is selected from a tracrRNA, and/or a crRNA, and/or an sgRNA, and/or a derivative of the tracrRNA, and/or a derivative of the crRNA, and/or a derivative of the sgRNA, and/or a guide DNA, and/or a guide RNA; the component (b) includes a Cas protein, and/or a derivative of the Cas protein, and/or an Ago protein, and/or a derivative of the Ago protein; and the component (a) and the component (b) are capable of binding with each other.

Preferably, the length of the mismatched nucleotide fragment is 0 bp, 1 bp, 2 bp, 3 bp, or 4 bp.

Preferably, the component (a) carries a marker, where the marker includes streptavidin or biotin.

More preferably, the component (a) carries a chemical modification, where the chemical modification includes a sulfur-substituted oxygen or methoxy modification.

More preferably, the sgRNA in the component (a) includes the mismatched nucleotide fragment.

More preferably, a length of a spacer sequence of the sgRNA in the component (a) is 16 nt-22 nt.

Furthermore preferably, sequences of the sgRNA are as follows:

(SEQ ID NO: 1)
UCUUAAUUCCUUGAUAGCGA;
(SEQ ID NO: 2)
UAGCUACAGUGAACUCUCGA;
(SEQ ID NO: 3)
UAGCUACAGUGAAAUCACGA;
(SEQ ID NO: 4)
GCUACAGUGAACUCUCGA;
(SEQ ID NO: 5)
GUCUAGCUACAGUGAAA;
(SEQ ID NO: 6)
GUCUAGCUGCAGUGAAA;
(SEQ ID NO: 27)
GGCAGCCGAAGGGCAUGAGC;
(SEQ ID NO: 28)
GGCAGCCGAAGAGCAUGAGC;
(SEQ ID NO: 37)
AAUUUUUGUUUGAGUGGUUG;
(SEQ ID NO: 42)
UCCAGCUGUAUCCAGUAUGU.

More preferably, sequences of the guide DNA are as follows:

forward guide:
(SEQ ID NO: 43)
p-TAGATTTCACTGTAGC-3';
and
reverse guide:
(SEQ ID NO: 44)
p-TTCTAGCTACAGTGAA-3'.

Preferably, the component (b) includes at least one of the following: Cas12a, SacCas9, CjCas9, SpCas9, NmCas9, Sp-Cas9 HF1, evoCas9, HypaCas9, HiFi Cas9, Sniper-Cas9, xCas9, eSpCas9 1.1, SuperFi Cas9, SaCas9, SaCas9-IF, efSaCas9, ScCas9, Cas9-Sc++, Cas9 HiFi-Sc++, SpaCas9, SpaCas9-IF, FnCas9, AnaCas9, SpyCas9, FnCas12a, LbCas12a, AsCas12a, CbAgo, TtAgo, PfAgo, KmAgo, or KpAgo.

Furthermore preferably, the Cas12a is from at least one of the following species: Francisella novicida; Acidaminococcus; or Lachnospiraceae.

Furthermore preferably, the SacCas9 is from the species Staphylococcus aureus.

Furthermore preferably, the CjCas9 is from the species Campylobacter jejuni.

Furthermore preferably, the SpCas9 is from the species Streptococcus pyogenes.

Furthermore preferably, the NmCas9 is from the species Neisseria meningitidis.

More preferably, the SaCas9 is from the species Staphylococcus aureus.

More preferably, the ScCas9 is from the species Streptococcus canis.

More preferably, the SpaCas9 is from the species Streptococcus pasteurianus.

More preferably, a final concentration of the component (a) in the ribonucleoprotein complex is 0.1 μM-2 μM.

More preferably, a final concentration of the component (b) in the ribonucleoprotein complex is 0.1 μM-3 μM.

More preferably, the ribonucleoprotein complex further includes a hydrogen ion buffer.

Furthermore preferably, a final concentration of the hydrogen ion buffer in the ribonucleoprotein complex is 1 mM-2 M.

Preferably, the kit further includes a constant-temperature cleavage-amplification reaction system.

Preferably, a final concentration of the ribonucleoprotein complex in the reaction system is 0.1 μM-2 μM.

Preferably, the reaction system further includes a primer, a gene of interest, an enzyme, and an auxochrome.

Preferably, the reaction system further includes deoxy-ribonucleoside triphosphate (dNTP) and a single-stranded binding protein.

More preferably, the gene of interest includes a genomic DNA or a cell-free DNA.

Furthermore preferably, the gene of interest includes the genomic DNA extracted from at least one of the following cell lines:
an EGFR 19del wild-type cell line; an EGFR 19 E746_A750 del (2235-2249del) cell line; an EGFR 19 E746_A750 del (2236-2250del) cell line; a BRAF V600E mutant cell line; a BRAF V600E wild-type cell line; or a B-CPAP cell line.

More preferably, the primer includes a nucleotide sequence capable of binding with the gene of interest and guiding synthesis.

Furthermore preferably, the primer includes at least one of the following sequences:

(SEQ ID NO: 15)
GCATGTGGCACCATCTCACA;
(SEQ ID NO: 16)
AGAGCAGCTGCCAGACATGA;
(SEQ ID NO: 19)
CTACACCTCAGATATATTTC;
(SEQ ID NO: 20)
TGGATCCAGACAACTGT;
(SEQ ID NO: 23)
TACGTGATGGCCAGCGTGGA;
(SEQ ID NO: 24)
ACTGGGAGCCAATATTGT;
(SEQ ID NO: 33)
TCGTTAAATAGATACGTTACGC;
(SEQ ID NO: 34)
TAAAAACTAAAAACTTTCCGCG;
(SEQ ID NO: 35)
TCGTTAAATAGATACGTTACGC;
(SEQ ID NO: 36)
CAACGCCTCGAAACCTACG;
(SEQ ID NO: 38)
CCCCCAGGATTCTTACAGAAAACAAGTGGT;
(SEQ ID NO: 39)
GCAAATACACAGAGGAAGCCTTCGCCTGTCCTC;
(SEQ ID NO: 40)
CAAGTGGTTATAGATGGTGA;
or,
(SEQ ID NO: 41)
CGCCTGTCCTCATGTATTGG

More preferably, the enzyme includes a DNA polymerase and/or a recombinase.

More preferably, the auxochrome includes MgOAc.

Preferably, the above reaction system may be configured to enrich mutant alleles having a minor allele frequency (MAF)≥0.01%.

A kit based method for detecting and/or enriching low-abundance mutant genes is further disclosed in the present disclosure. The method includes:
collecting a sample;
preparing a ribonucleoprotein complex;
preparing a reaction system; and
performing enrichment analysis.

Preferably, the step of collecting a sample includes extracting a DNA and/or an RNA from a sample through a nucleic acid extraction kit.

More preferably, the sample includes at least one of the following: blood, plasma/serum, a cerebrospinal fluid, urine, or saliva.

Furthermore preferably, the sample includes at least one of the following obtained from a cancer patient: blood, plasma/serum, a cerebrospinal fluid, urine, or saliva.

Preferably, the step of preparing a ribonucleoprotein complex includes: mixing and incubating Cas9, sgRNA, and a hydrogen ion buffer.

Preferably, the hydrogen ion buffer includes HEPES.

Preferably, in preparation of the ribonucleoprotein complex, a final concentration of the Cas9 used is 0.1 μM-3 μM.

More preferably, in the preparation of the ribonucleoprotein complex, the final concentration of the Cas9 used is 2.5 μM.

Preferably, in the preparation of the ribonucleoprotein complex, a final concentration of the sgRNA used is 0.1 μM-2 μM.

More preferably, in the preparation of the ribonucleoprotein complex, the final concentration of the sgRNA used is 2.5 μM.

Preferably, in the preparation of the ribonucleoprotein complex, a final concentration of the HEPES used is 1 mM-2 M.

More preferably, in the preparation of the ribonucleoprotein complex, the final concentration of the HEPES used is 1 M.

Preferably, in the preparation of the ribonucleoprotein complex, a reaction temperature is 34° C.-39° C., and a reaction time is 3 min-18 min.

More preferably, in the preparation of the ribonucleoprotein complex, the reaction temperature is 37° C., and the reaction time is 10 min.

Preferably, the step of preparing a reaction system includes preparing a constant-temperature cleavage-amplification reaction system.

More preferably, a final concentration of the ribonucleoprotein complex in the reaction system is 0.1 μM-2 μM.

More preferably, the reaction system further includes a primer, a gene of interest, an enzyme, and an auxochrome.

More preferably, the reaction system further includes dNTP and a single-stranded binding protein.

Furthermore preferably, the gene of interest includes a genomic DNA.

Furthermore preferably, the gene of interest includes the genomic DNA extracted from at least one of the following cell lines:
an EGFR 19del wild-type cell line; an EGFR 19 E746_A750 del (2235-2249del) cell line; an EGFR 19 E746_A750 del (2236-2250del) cell line; a BRAF V600E mutant cell line; a BRAF V600E wild-type cell line; or a B-CPAP cell line.

Furthermore preferably, the primer includes a nucleotide sequence capable of binding with the gene of interest and guiding synthesis.

Furthermore preferably, the primer includes at least one of the following sequences:

(SEQ ID NO: 15)
GCATGTGGCACCATCTCACA;
(SEQ ID NO: 16)
AGAGCAGCTGCCAGACATGA;
(SEQ ID NO: 19)
CTACACCTCAGATATATTTC;
(SEQ ID NO: 20)
TGGATCCAGACAACTGT;
(SEQ ID NO: 23)
TACGTGATGGCCAGCGTGGA;
(SEQ ID NO: 24)
ACTGGGAGCCAATATTGT;
(SEQ ID NO: 33)
TCGTTAAATAGATACGTTACGC;
(SEQ ID NO: 34)
TAAAAACTAAAAACTTTCCGCG;
(SEQ ID NO: 35)
TCGTTAAATAGATACGTTACGC;
(SEQ ID NO: 36)
CAACGCCTCGAAACCTACG;
(SEQ ID NO: 38)
CCCCCAGGATTCTTACAGAAAACAAGTGGT;
(SEQ ID NO: 39)
GCAAATACACAGAGGAAGCCTTCGCCTGTCCTC;
(SEQ ID NO: 40)
CAAGTGGTTATAGATGGTGA;
or,
(SEQ ID NO: 41)
CGCCTGTCCTCATGTATTGG.

Furthermore preferably, the enzyme includes a DNA polymerase and/or a recombinase.

Furthermore preferably, the auxochrome includes MgOAc.

Preferably, the step of performing enrichment analysis includes QPCR analysis, sequencing, point of care testing (POCT), and the above programmable nuclease based method for detecting a low-abundance mutant gene.

More preferably, the step of QPCR analysis includes preparing a QPCR system.

Furthermore preferably, the QPCR system includes:
a primer, an auxochrome, dNTP, an enzyme.

Furthermore preferably, the primer includes at least one of the following:

(SEQ ID NO: 17)
TGTCATAGGGACTCTGGATCCCAGA;
(SEQ ID NO: 18)
GCAGAAACTCACATCGAGGATTTCCTTGT;
(SEQ ID NO: 21)
CCTCAGATATATTTCTTCATGA;
(SEQ ID NO: 22)
TGTTCAAACTGATGGGAC;
(SEQ ID NO: 25)
TGATGGCCAGCGTGGACAA;
or,
(SEQ ID NO: 26)
TTGTGTTCCCGGACATAGTC.

Furthermore preferably, the auxochrome includes MgOAc.

Furthermore preferably, the enzyme includes a DNA polymerase.

A CRISPR system based method for designing an sgRNA is further disclosed in the present disclosure. The sgRNA includes a specific recognition nucleotide fragment and a mismatched nucleotide fragment, where a length of the mismatched nucleotide fragment is 0 bp-4 bp.

Preferably, the specific recognition nucleotide fragment is a spacer sequence.

More preferably, a length of the spacer sequence is 18 nt-22 nt.

Preferably, the length of the mismatched nucleotide fragment is 0 bp, 1 bp, 2 bp, 3 bp, or 4 bp.

Through the sgRNA designed through the method for designing an sgRNA of the present disclosure, a wild-type allele and a mutant allele can be effectively distinguished from each other. Moreover, the spacer sequence of the sgRNA designed through the method for designing an sgRNA of the present disclosure has high recognition and cleavage efficiency. Through the mismatched nucleotide fragment designed by the present disclosure, the application range of the sgRNA can be expanded, and more mutations can be recognized by the sgRNA.

An sgRNA is further disclosed in the present disclosure. The sgRNA includes a specific recognition nucleotide fragment and a mismatched nucleotide fragment, where a length of the mismatched nucleotide fragment is 0 bp-4 bp.

Preferably, the length of the mismatched nucleotide fragment is 0 bp, 1 bp, 2 bp, 3 bp, or 4 bp.

Preferably, the above sgRNA is designed through the above method.

Preferably, a site of the mismatched nucleotide is located at 5 bps in front of a mutant gene and/or 5 bps behind the mutant gene.

Preferably, the specific recognition nucleotide fragment is a spacer sequence.

More preferably, a length of the spacer sequence is 18 nt-22 nt.

More preferably, sequences of the sgRNA are as follows:

(SEQ ID NO: 1)
UCUUAAUUCCUUGAUAGCGA;
(SEQ ID NO: 2)
UAGCUACAGUGAACUCUCGA;
(SEQ ID NO: 3)
UAGCUACAGUGAAAUCACGA;
(SEQ ID NO: 4)
GCUACAGUGAACUCUCGA;
(SEQ ID NO: 5)
GUCUAGCUACAGUGAAA;
(SEQ ID NO: 6)
GUCUAGCUGCAGUGAAA;
(SEQ ID NO: 27)
GGCAGCCGAAGGGCAUGAGC;
(SEQ ID NO: 28)
GGCAGCCGAAGAGCAUGAGC;
(SEQ ID NO: 37)
AAUUUUUGUUUGAGUGGUUG;
(SEQ ID NO: 42)
UCCAGCUGUAUCCAGUAUGU.

Use of the above sgRNA is further disclosed in the present disclosure. The use includes at least one of the following:
(1) enriching gene mutation;
(2) detecting gene mutation;
(3) enriching methylated DNAs; or
(4) detecting methylated DNAs.

A guide DNA or a guide RNA is further disclosed in the present disclosure. The guide DNA or the guide RNA includes a specific recognition nucleotide fragment and a mismatched nucleotide fragment, where a length of the mismatched nucleotide fragment is 0 bp-4 bp.

Preferably, the length of the mismatched nucleotide fragment is 0 bp, 1 bp, 2 bp, 3 bp, or 4 bp.

Preferably, the above guide DNA includes sequences as follows:

SEQ ID NO: 43: forward guide:
p-TAGATTTCACTGTAGC-3';
and
SEQ ID NO: 44: reverse guide:
p-TTCTAGCTACAGTGAA-3'.

Use of the above guide DNA or the above guide RNA is further disclosed in the present disclosure. The use includes at least one of the following:
(1) enriching gene mutation;
(2) detecting gene mutation;
(3) enriching methylated DNAs; or
(4) detecting methylated DNAs.

A programmable enzyme based method for enriching mutant genes and/or methylated DNAs is further disclosed in the present disclosure. A CRISPR system is configured to specifically cleave a wild-type allele and amplify a mutant allele. The CRISPR system includes:
a component (a), which includes a nucleic acid region capable of complementarily matching a target nucleic acid and 0 bp-4 bp mismatched fragments; and
a component (b), which is capable of binding with the target nucleic acid and breaking a strand of the target nucleic acid;
where the component (a) is selected from a tracrRNA, and/or a crRNA, and/or an sgRNA, and/or a derivative of the tracrRNA, and/or a derivative of the crRNA, and/or a derivative of the sgRNA, and/or a guide DNA, and/or a guide RNA; the component (b) includes a Cas protein, and/or a derivative of the Cas protein, and/or an Ago protein, and/or a derivative of the Ago protein; and the component (a) and the component (b) are capable of binding with each other. Preferably, the component (a) carries a marker, where the marker includes streptavidin or biotin.

Preferably, the length of the mismatched nucleotide fragment is 0 bp, 1 bp, 2 bp, 3 bp, or 4 bp.

Preferably, the component (a) carries a chemical modification, where the chemical modification includes a sulfur-substituted oxygen or methoxy modification.

Preferably, the sgRNA in the component (a) includes the mismatched nucleotide fragment. Through the mismatched nucleotide fragment designed by the present disclosure, the application range of the sgRNA can be expanded, and more mutations can be recognized by the sgRNA.

Preferably, a length of a spacer sequence of the sgRNA in the component (a) is 16 nt-22 nt.

Preferably, the component (b) includes at least one of the following: Cas12a, SacCas9, CjCas9, SpCas9, NmCas9, Sp-Cas9 HF1, evoCas9, HypaCas9, HiFi Cas9, Sniper-Cas9, xCas9, eSpCas9 1.1, SuperFi Cas9, SaCas9, SaCas9-IF, efSaCas9, ScCas9, Cas9-Sc++, Cas9 HiFi-Sc++, SpaCas9, SpaCas9-IF, FnCas9, AnaCas9, SpyCas9, FnCas12a, LbCas12a, AsCas12a, CbAgo, TtAgo, PfAgo, KmAgo, or KpAgo.

More preferably, the Cas12a is from at least one of the following species: Francisella novicida; Acidaminococcus; or Lachnospiraceae.

More preferably, the SacCas9 is from the species Staphylococcus aureus.

More preferably, the CjCas9 is from the species Campylobacter jejuni.

More preferably, the SpCas9 is from the species Streptococcus pyogenes.

More preferably, the NmCas9 is from the species Neisseria meningitidis.

More preferably, the SaCas9 is from the species Staphylococcus aureus.

More preferably, the ScCas9 is from the species Streptococcus canis.

More preferably, the SpaCas9 is from the species Streptococcus pasteurianus.

Preferably, a final concentration of the component (a) in the CRISPR system is 0.1 μM-2 μM.

Preferably, a final concentration of the component (b) in the CRISPR system is 0.1 μM-3 μM.

Preferably, the CRISPR system further includes a hydrogen ion buffer.

More preferably, the hydrogen ion buffer in the CRISPR system includes HEPES.

More preferably, a final concentration of the hydrogen ion buffer in the CRISPR system is 1 mM-2 M.

Preferably, a method for amplifying a mutant allele includes PCR, loop-mediated isothermal amplification (LAMP), and recombinase polymerase amplification (RPA).

More preferably, a reaction system for amplifying a mutant allele includes a ribonucleoprotein complex.

Preferably, a final concentration of the ribonucleoprotein complex in the reaction system is 0.1 μM-2 μM.

Preferably, the reaction system further includes a primer, a gene of interest, an enzyme, and an auxochrome.

Preferably, the reaction system further includes deoxy-ribonucleoside triphosphate (dNTP) and a single-stranded binding protein.

More preferably, the gene of interest includes a genomic DNA.

Furthermore preferably, the gene of interest includes the genomic DNA extracted from at least one of the following cell lines:
an EGFR 19del wild-type cell line; an EGFR 19 E746_A750 del (2235-2249del) cell line; an EGFR 19 E746_A750 del (2236-2250del) cell line; a BRAF V600E mutant cell line; a BRAF V600E wild-type cell line; or a B-CPAP cell line.

More preferably, the primer includes a nucleotide sequence capable of binding with the gene of interest and guiding synthesis.

Furthermore preferably, the primer includes at least one of the following sequences:

(SEQ ID NO: 15)
GCATGTGGCACCATCTCACA;
(SEQ ID NO: 16)
AGAGCAGCTGCCAGACATGA;
(SEQ ID NO: 19)
CTACACCTCAGATATATTTC;
(SEQ ID NO: 20)
TGGATCCAGACAACTGT;
(SEQ ID NO: 23)
TACGTGATGGCCAGCGTGGA;
(SEQ ID NO: 24)
ACTGGGAGCCAATATTGT;
(SEQ ID NO: 33)
TCGTTAAATAGATACGTTACGC;
(SEQ ID NO: 34)
TAAAAACTAAAAACTTTCCGCG;
(SEQ ID NO: 35)
TCGTTAAATAGATACGTTACGC;
(SEQ ID NO: 36)
CAACGCCTCGAAACCTACG;
(SEQ ID NO: 38)
CCCCCAGGATTCTTACAGAAAACAAGTGGT;
(SEQ ID NO: 39)
GCAAATACACAGAGGAAGCCTTCGCCTGTCCTC;
(SEQ ID NO: 40)
CAAGTGGTTATAGATGGTGA;
or,
(SEQ ID NO: 41)
CGCCTGTCCTCATGTATTGG.

More preferably, sequences of the guide DNA are as follows:

forward guide:
(SEQ ID NO: 43)
p-TAGATTTCACTGTAGC-3';
and
reverse guide:
(SEQ ID NO: 44)
p-TTCTAGCTACAGTGAA-3'.

More preferably, the enzyme includes a DNA polymerase and/or a recombinase.

More preferably, the auxochrome includes MgOAc.

Furthermore preferably, the ribonucleoprotein complex includes Cas9, the sgRNA, and the hydrogen ion buffer.

Furthermore preferably, a method for preparing a ribonucleoprotein complex includes: mixing and incubating the Cas9, the sgRNA, and the hydrogen ion buffer.

Furthermore preferably, the hydrogen ion buffer includes HEPES.

Furthermore preferably, in preparation of the ribonucleoprotein complex, a final concentration of the Cas9 used is 0.1 μM-3 μM.

Furthermore preferably, in the preparation of the ribonucleoprotein complex, a final concentration of the sgRNA used is 0.1 μM-2 μM.

Furthermore preferably, in the preparation of the ribonucleoprotein complex, a final concentration of the HEPES used is 1 mM-2 M.

Furthermore preferably, in the preparation of the ribonucleoprotein complex, a reaction temperature is 34° C.-39° C., and a reaction time is 3 min-18 min.

A programmable nuclease based method for detecting a low-abundance mutant gene and/or a methylated DNA is further disclosed in the present disclosure. The above CRISPR system or an Ago system is configured to specifically cleave a wild-type allele and amplify a mutant allele. The method includes:
designing an sgRNA;
acquiring a gene of interest;
performing amplification and cleavage; and
detecting the mutant gene.

Preferably, the sgRNA includes a mismatched nucleotide fragment.

Preferably, a length of a spacer sequence of the sgRNA is 16 nt-22 nt.

More preferably, the gene of interest includes: EGFR, BRAF, PIK3CA, TP53, LRP1B, APC, CYP1A1, NPO1, EPHX1, KRAS, BRCA1, BRCA2, MET, MLH1, MSH2, MSH3, MSH6, PALB2, BMPR1A, SMAD4, STK11, PTEN, AXIN2, BLM, BUB1B, CDH1, CEP57, CHEK2, ENG, EPCAM, FLCN, GALNTI2, GREM1, FAT4, KMT2D, KMT2C, ARID1A, FAT1, PTEN, ATM, ZFHX3, CREBBP, GRIN2A, NRAS, or NF1.

Preferably, an amplification method includes PCR, LAMP, and RPA.

Preferably, the method for detecting a mutant gene includes sanger sequencing.

Preferably, a reaction system used for amplification includes the gene of interest, a ribonucleoprotein complex, MgOAc, a primer, a DNA polymerase, a recombinase, and a single-stranded binding protein.

Preferably, a reaction system used for cleavage includes the gene of interest, a ribonucleoprotein complex, and MgOAc.

A microfluidic chip for automatically enriching and/or detecting mutant genes and/or methylated DNAs is further disclosed in the present disclosure.

Preferably, the microfluidic chip is divided into three parts and consists of a top package sheet, a bottom package sheet, and an intermediate reaction layer.

More preferably, a structure of the top package sheet includes a microfluidic chip mounting hole; a microfluidic chip package positioning hole; and a microfluidic chip sample feeding hole.

More preferably, a structure of the intermediate reaction layer includes a microfluidic chip package positioning hole; a microfluidic chip mounting hole; a pre-amplification reaction chamber; a siphon valve; a digestion reaction chamber; a pre-distribution chamber; a PCR chamber; a waste liquid chamber; a capillary valve; and a gas passage.

More preferably, a structure of the bottom package sheet includes a microfluidic chip mounting hole; a microfluidic chip package positioning hole; an RNase A adding chamber; and a proteinase K adding chamber.

A method for automatically enriching and/or detecting mutant genes and/or methylated DNAs is further disclosed in the present disclosure. The method includes:
1) adding a sample: a pre-amplification system is added into a pre-amplification reaction chamber; RNase A is added into the RNase A adding chamber; proteinase K is added into the proteinase K adding chamber; and a qPCR system is added into a PCR chamber;
2) sealing; and
3) loading and experimenting: a sealed microfluidic chip is loaded on a centrifugal microfluidic platform.

Compared with the related art, the present disclosure has the beneficial effects as follows: according to the present disclosure, based on the CRISPR system, the programmable nuclease, and the nucleic acid amplification technology, the wild-type nucleic acid is specifically cleaved through the programmable nuclease while amplified. Thus, the number of mutant alleles in the sample continuously increases exponentially and reaches a level detectable through low-cost sanger sequencing. The detection sensitivity comparable to ultra-deep next-generation sequencing (NGS) is achieved. Moreover, a detection cycle is short, and no large-scale instrument and apparatus is required. Thus, a detection cost is greatly reduced, and the clinical application of low-abundance mutant allele detection is significantly promoted. Through the enrichment method of the present disclosure, the mutant alleles having the MAF≥0.01% can be enriched, and a frequency of the mutant alleles can be obviously increased.

### BRIEF DESCRIPTION OF THE DRAWINGS

FIG. 1 is a schematic diagram of enriching low-abundance mutant alleles according to the present disclosure;

FIG. 2 is a diagram of a cleavage verification result of a single guide ribonucleic acid (sgRNA) in Example 1;

FIG. 3 shows sequencing results of a wild-type allele and ED2 before and after treatment of ED2 with SpCas9 in Example 1;

FIG. 4 shows a sequencing result of ED2 after treatment of ED2 with Hypa Cas9 in Example 1;

FIG. 5 shows sequencing results of a wild-type allele and ED1 before and after treatment of ED1 with SpCas9 HF1 in Example 1;

FIG. 6 shows a sequencing result of ED1 after treatment of ED1 with Evo Cas9 in Example 1;

FIG. 7 is a diagram of a cleavage verification result of an sgRNA in Example 2;

FIG. 8 shows sequencing results of a wild-type allele and a mutant allele before and after treatment with HiFi Cas9 in Example 2;

FIG. 9 shows a sequencing result of a mutant allele after treatment with SuperFi Cas9 in Example 2;

FIG. 10 shows a sequencing result of a mutant allele after treatment with HiFi SC++ for 0 min in Example 2;

FIG. 11 shows a sequencing result of a mutant allele after treatment with HiFi SC++ for 5 min in Example 2;

FIG. 12 shows a sequencing result of a mutant allele after treatment with HiFi SC++ for 10 min in Example 2;

FIG. 13 shows a sequencing result of a mutant allele after treatment with HiFi SC++ for 15 min in Example 2;

FIG. 14 shows a sequencing result of a mutant allele after treatment with HiFi SC++ for 20 min in Example 2;

FIG. 15 is a diagram of a cleavage verification result of an sgRNA in Example 3;

FIG. 16 shows sequencing results of a wild-type allele and a mutant allele before and after treatment with Cas9 in Example 3;

FIG. 17 shows a comparison of enrichment degrees of a 0.1% methylated deoxyribonucleic acid (DNA) before and after enrichment treatment in Example 4;

FIG. 18 is a schematic diagram of enriching methylated DNAs according to the present disclosure;

FIG. 19 shows sequencing results of a wild-type allele and a mutant allele before and after treatment of an NRAS with Cas12 in Example 5;

FIG. 20 is a structural diagram of a microfluidic chip;

FIG. 21 is a structural diagram of a top package sheet;

FIG. 22 is a structural diagram of an intermediate traction layer;

FIG. 23 is a structural diagram of a bottom package sheet;

FIG. 24 is a schematic diagram based on argonaute (Ago) cleavage according to the present disclosure; and

FIG. 25 shows a comparison of results before and after treatment with programmable enzyme based selective exponential amplification (PASEA) in a case of a minor allele frequency (MAF)=0.1% in Example 7.

Reference numerals: 1—top package sheet;
11—microfluidic chip mounting hole; 12—microfluidic chip package positioning hole; and 13—microfluidic chip sample feeding hole;
2—intermediate reaction layer;
21—microfluidic chip package positioning hole; 22—microfluidic chip mounting hole; 23—pre-amplification reaction chamber; 24—siphon valve; 25—digestion reaction chamber; 26—pre-distribution chamber; 27—polymerase chain reaction (PCR) chamber; 28—waste liquid chamber; 29—capillary valve; and 210—gas passage; and
3—bottom package sheet;
31—microfluidic chip mounting hole; 32—microfluidic chip package positioning hole; 33—RNase A adding chamber; and 34—proteinase K adding chamber.

### DETAILED DESCRIPTION OF THE EMBODIMENTS

Illustrative examples are described in detail herein. Embodiments described in the following illustrative examples do not denote all embodiments consistent with the present disclosure. Rather, the embodiments are merely instances of methods consistent with some aspects of the present disclosure.

The experimental methods in the following examples are conventional methods or performed according to the conditions recommended by the manufacturer, unless specified otherwise. The materials, reagents, etc. used in the following examples are commercially available, unless specified otherwise.

A primer used for sequencing in the following examples is identical to a primer for a quantitative polymerase chain reaction (qPCR).

### Example 1

Enrichment of EGFR gene del19 mutations

### 1, Primers are Designed.

A gene sequence of an exon 19 of a human epidermal growth factor receptor (EGFR) gene is acquired from the national center for biotechnology information (NCBI) database, and the primers are designed according to the sequence.

19del pre-amplification F primer:
(sequence identifier number 15; (SEQ ID NO: 15))
GCATGTGGCACCATCTCACA;
19del pre-amplification R primer:
(SEQ ID NO: 16)
AGAGCAGCTGCCAGACATGA;
19del qPCR F primer:
(SEQ ID NO: 17)
TGTCATAGGGACTCTGGATCCCAGA;
and
19del qPCR R primer:
(SEQ ID NO: 18)
GCAGAAACTCACATCGAGGATTTCCTTGT

2, a Single Guide Ribonucleic Acid (sgRNA) is Designed.

An sgRNA sequence having a length of 20 nt and capable of completely matching a wild-type allele sequence is designed as shown in SEQ ID NO: 1:

(SEQ ID NO: 1)
UCUUAAUUCCUUGAUAGCGA

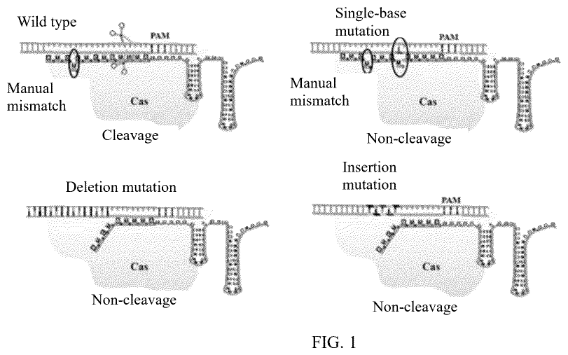

The sgRNA is configured to guide a clustered regularly interspaced short palindromic repeats (CRISPR)-associated (Cas) protein to bind with a wild-type allele and specifically cleave the wild-type allele rather than a mutant allele. The principle is as shown in FIG. 1.

### 3, Genes of Interest are Acquired.

Gene short-chain sequences used as markers in the example are shown as follows:

EGFR-WT-S:
(SEQ ID NO: 7)
CTCTGGATCCCAGAAGGTGAGAAAGTTAAAATTCCCGTCGCTATC
AAGGAATTAAGAGAAGCAACATCTCCGAAAGCCAACAAGGAAATC
CTCGATGTGA;
EGFR-WT-AS:
(SEQ ID NO: 8)
TCACATCGAGGATTTCCTTGTTGGCTTTCGGAGATGTTGCTTCTC
TTAATTCCTTGATAGCGACGGGAATTTTAACTTTCTCACCTTCTG
GGATCCAGAG;
EGFR-del1-S:
(SEQ ID NO: 9)
CTCTGGATCCCAGAAGGTGAGAAAGTTAAAATTCCCGTCGCTATC
AAAACATCTCCGAAAGCCAACAAGGAAATCCTCGATGTGAGTTTC
TGCTTTGCTG;
EGFR-del1-AS:
(SEQ ID NO: 10)
CAGCAAAGCAGAAACTCACATCGAGGATTTCCTTGTTGGCTTTCG
GAGATGTTTTGATAGCGACGGGAATTTTAACTTTCTCACCTTCTG
GGATCCAGAG;
EGFR-del2-S:
(SEQ ID NO: 11)
CTCTGGATCCCAGAAGGTGAGAAAGTTAAAATTCCCGTCGCTATC
AAGACATCTCCGAAAGCCAACAAGGAAATCCTCGATGTGAGTTTC
TGCTTTGCTG;
EGFR-del2-AS:
(SEQ ID NO: 12)
CAGCAAAGCAGAAACTCACATCGAGGATTTCCTTGTTGGCTTTCG
GAGATGTCTTGATAGCGACGGGAATTTTAACTTTCTCACCTTCTG
GGATCCAGAG;
EGFR-del3-S:
(SEQ ID NO: 13)
TCTGGATCCCAGAAGGTGAGAAAGTTAAAATTCCCGTCGCTATCA
AGGAATCGAAAGCCAACAAGGAAATCCTCGATGTGAGTTTCTGCT
TTGCTGTGTG;
EGFR-del3-AS:
(SEQ ID NO: 14)
CACACAGCAAAGCAGAAACTCACATCGAGGATTTCCTTGTTGGCT
TTCGATTCCTTGATAGCGACGGGAATTTTAACTTTCTCACCTTCT
GGGATCCAGA;

the above sequences are synthesized by Sangon Biotech (Shanghai) Co., Ltd.

The EGFR 19del wild-type allele is sourced from a B-CPAP cell line; and the mutant alleles EGFR 19 E746_A750 del (2235-2249del) and EGFR 19 E746_A750 del (2236-2250del) are sourced from an NCI-H1650 cell line and an HCC827 cell line respectively. The cell lines are purchased from Shanghai Zhongqiaoxinzhou Biotech Co., Ltd. Genomic deoxyribonucleic acids (DNAs) of two cell lines are extracted through a QIAGEN DNeasy Blood & Tissue Kit according to the instructions. Two genomic DNAs are prepared into templates having minor allele frequencies (MAFs)=5%, 1%, 0.5%, 0.1%, and 0.01% for enrichment and detection of mutations.

4, Cleavage Verification of the sgRNA is Performed.

### a) Components of a Ribonucleoprotein Complex Shown in Table 1

TABLE 1
Ribonucleoprotein Complex

| | Final Concentration |
|---|---|
| Cas9 | 2.5 μm |
| sgRNA | 2.5 μm |
| HEPES | 1 M |

A Cas protein herein includes one of SpCas9 HF1 (configured for enrichment and detection of ED1), Evo Cas9 (configured for enrichment and detection of ED1), SpCas9 (configured for enrichment and detection of ED2), or Hypa Cas9 (configured for enrichment and detection of ED2).

Incubation is performed at a constant temperature 37° C. for 10 min.

### b) Constituents of a Cleavage System Shown in Table 2

TABLE 2
Cleavage System

| | Final Concentration |
|---|---|
| Ribonucleoprotein Complex | 2.5 μm |
| MgOAc | 14 mM |
| Template | 0.25 μm |

Cleavage is performed at a constant temperature 37° C. for 1 h.

### c) Digestion

First, 1 μl of RNase A (10 mg/ml) is added and stood at a room temperature for 10 min. Then, 1 μl of proteinase K (20 mg/ml) is added, and a constant temperature 56° C. is maintained for 30 min. Finally, a constant temperature 95° C. is maintained for 10 min.

### d) Gel Running

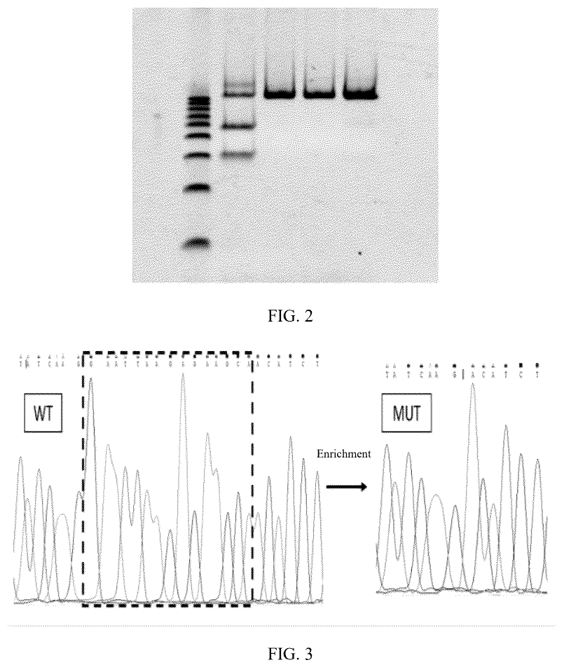

Polyacrylamide gel electrophoresis (PAGE) is performed. A loading buffer is added into a cleaved product. A sample is added. Electrophoresis is performed at 150 V for 10 min. After the electrophoresis is ended, staining with a gelred dye is performed for 15 min; and then a photo is taken (see FIG. 2). The five lanes in FIG. 2 denote the marker, WT, ED1, ED2, and ED3 from left to right. ED1 denotes the E746_A750 del (2235-2249del) mutation. ED2 denotes the EGFR 19 E746_A750 del (2236-2250del) mutation. ED3 denotes the EGFR 19 E746_A750 del (2240-2257del) mutation. As can be seen in FIG. 2, the wild-type allele is cleaved under the guide of the sgRNA, and almost no other mutant alleles are cleaved.

### 5, Mutant Alleles are Enriched and Detected.

The mutations ED1 and ED2 are enriched and detected in this step.

### a) Pre-Amplification:

Components of a ribonucleoprotein complex are shown in Table 3.

TABLE 3
Ribonucleoprotein Complex

| | Final Concentration |
|---|---|
| Cas9 | 1 μm |
| sgRNA | 1 μm |
| HEPES | 1 M |

A Cas protein herein includes one of SpCas9 HF1 (configured for enrichment and detection of ED1), Evo Cas9 (configured for enrichment and detection of ED1), SpCas9 (configured for enrichment and detection of ED2), or Hypa Cas9 (configured for enrichment and detection of ED2). During specific operation, the Cas protein used for preparing the ribonucleoprotein complex in this step corresponded to the Cas9 used for the cleavage verification of the sgRNA in step 4.

Incubation is performed at a constant temperature 37° C. for 10 min, to obtain the ribonucleoprotein complex.

Constituents of a pre-amplification system are shown in Table 4.

TABLE 4
Pre-amplification System

| | Final Concentration |
|---|---|
| Ribonucleoprotein Complex | 1 μm |
| Pre-amplification F primer | 0.5 μm |
| Pre-amplification R primer | 0.5 μm |
| MgOAc | 14 mM |
| dNTP | 0.45 mM |
| DNA polymerase | 30 ng/μl |
| Recombinase | 900 ng/μl |
| Single-stranded DNA-binding | 150 ng/μl |
| protein (SSB) | |
| Template | 60 ng |

A reaction is performed at a constant temperature 37° C. for 20 min; and then the reaction is performed at 95° C. for 10 min and then terminated.

### Digestion:

First, 1 μl of RNase A (10 mg/ml) is added and stood at a room temperature for 10 min. Then, 1 μl of proteinase K (20 mg/ml) is added, and a constant temperature 56° C. is maintained for 30 min. Finally, a constant temperature 95° C. is maintained for 10 min.

Constituents of a qPCR system are shown in Table 5.

TABLE 5
qPCR System

| | Final Concentration |
|---|---|
| qPCR F primer | 1 μm |
| qPCR R primer | 1 μm |
| dNTP | 0.45 mM |
| DNA polymerase | 30 ng/μl |

### Reaction Procedure:

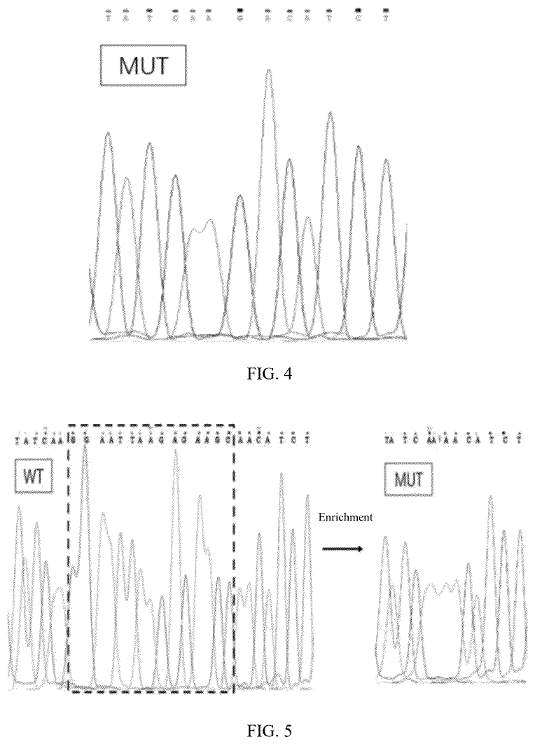

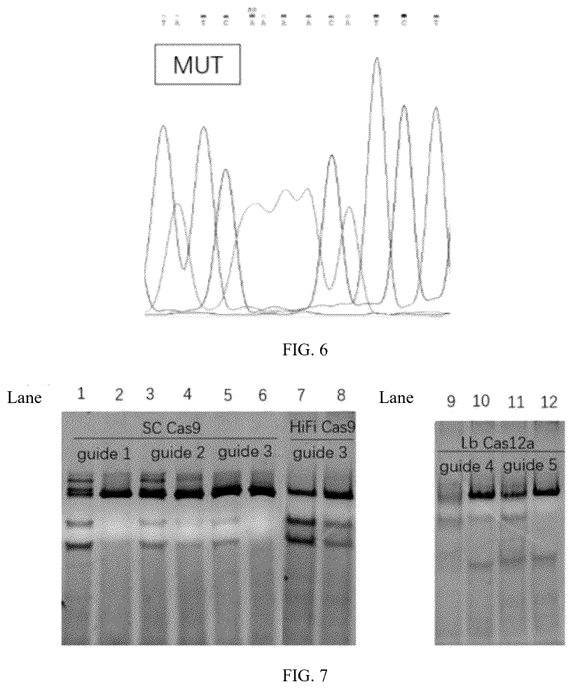

Pre-denaturation: 95° C., 5 min;
45 cycles;
95° C., 10 s;
60° C., 30 s; and
after amplification, sanger sequencing is performed on amplified products, and sequencing results are shown in FIGS. 3-9. To avoid redundancy, only enrichment results of samples having an MAF=0.1% are shown. As can be seen in FIGS. 3-9, wild-type alleles are increased significantly after enrichment. It indicates that the enrichment method of the present disclosure can effectively enrich the mutant alleles.

### Example 2

### Enrichment and Detection of BRAF Gene V600E Mutations

### 1, Primers are Designed.

A human BRAF gene sequence is acquired from the NCBI database, and the primers shown as follows are designed according to the sequence:

pre-amplification F primer:
(SEQ ID NO: 19)
CTACACCTCAGATATATTTC;
pre-amplification R primer:
(SEQ ID NO: 20)
TGGATCCAGACAACTGT;
qPCR F primer:
(SEQ ID NO: 21)
CCTCAGATATATTTCTTCATGA;
and
qPCR R primer:
(SEQ ID NO: 22)
TGTTCAAACTGATGGGAC

2, an sgRNA is Designed.

To be specific, sgRNA sequences having lengths of 17 nt-20 nt and capable of completely matching a wild-type allele sequence are designed as shown in SEQ ID NOS: 2-6:

guide 1:
(SEQ ID NO: 2)
UAGCUACAGUGAACUCUCGA
guide 2:
(SEQ ID NO: 3)
UAGCUACAGUGAAAUCACGA
guide 3:
(SEQ ID NO: 4)
GCUACAGUGAACUCUCGA
guide 4:
(SEQ ID NO: 5)
GUCUAGCUACAGUGAAA
guide 5:
(SEQ ID NO: 6)
GUCUAGCUGCAGUGAAA

### 3, Genomic DNAs are Acquired.

A BRAF V600E mutant allele and a BRAF V600E wild-type allele are sourced from a B-CPAP cell line and an HCC827 cell line (purchased from Shanghai Zhongqiaoxinzhou Biotech Co., Ltd) respectively. Genomic DNAs of two cell lines are extracted through a QIAGEN DNeasy Blood & Tissue Kit according to the instructions. Two DNAs are prepared into templates having MAFs=5%, 1%, 0.5%, 0.1%, and 0.01% for enrichment and detection of mutations.

4, Cleavage Verification of the sgRNA is Performed.

### a) Components of a Ribonucleoprotein Complex

The following reaction system is prepared as shown in Table 6.

TABLE 6
Reaction System

| | Final Concentration |
|---|---|
| Cas9 | 2.5 μm |
| sgRNA | 2.5 μm |
| HEPES | 1 M |

A Cas9 herein includes one of HiFi Cas9, SuperFi Cas9, or HiFi SC++.

Incubation is performed at a constant temperature 37° C. for 10 min, to obtain the ribonucleoprotein complex.

### b) Constituents of a Cleavage System Shown in Table 7.

TABLE 7
Cleavage System

| | Final Concentration |
|---|---|
| Ribonucleoprotein Complex | 2.5 μm |
| MgOAc | 14 mM |
| Template | 0.25 μm |

Cleavage is performed at a constant temperature 37° C. for 1 h.

### c) Digestion

First, 1 μl of RNase A (10 mg/ml) is added and stood at a room temperature for 10 min. Then, 1 μl of proteinase K (20 mg/ml) is added, and a constant temperature 56° C. is maintained for 30 min. Finally, a constant temperature 95° C. is maintained for 10 min.

### d) Gel Running

Polyacrylamide gel electrophoresis (PAGE) is performed. A loading buffer is added into a cleaved product. A sample is added. Electrophoresis is performed at 150 V for 10 min.

After the electrophoresis is ended, staining with a gelred dye is performed for 15 min, and a photo is taken (shown in FIG. 7). The lanes 1, 3, 5, 7, 9, and 11 denote wild-type nucleic acids, and the lanes 2, 4, 6, 8, 10, and 12 denote mutant nucleic acids. By comparing the lanes 1, 2, 3, 4, 5, 6, 7, 8, 9, 10, 11, and 12 separately, it can be seen that the wild-type alleles are more likely to be cleaved than the mutant alleles. By comparing the lanes 1, 3, 5, 7, 9, and 11, guide 3 hardly cleaved the wild-type alleles.

### 5, Mutant Alleles are Enriched and Detected.

### a) Pre-Amplification:

Components of a ribonucleoprotein complex are shown in Table 8.

TABLE 8
Ribonucleoprotein Complex

| | Final Concentration |
|---|---|
| Cas9 | 1 μm |
| sgRNA | 1 μm |
| HEPES | 1 M |

Incubation is performed at a constant temperature 37° C. for 10 min, to obtain a ribonucleoprotein complex.

Constituents of a pre-amplification system are shown in Table 9.

TABLE 9
Pre-amplification System

| | Final Concentration |
|---|---|
| Ribonucleoprotein Complex | 1 μm |
| Pre-amplification F primer | 0.5 μm |
| Pre-amplification R primer | 0.5 μm |
| MgOAc | 14 mM |
| dNTP | 0.45 mM |
| DNA polymerase | 30 ng/μl |
| Recombinase | 900 ng/μl |
| SSB | 150 ng/μl |
| Template | 60 ng |

A Cas9 herein includes one of HiFi Cas9, SuperFi Cas9, or HiFi SC++. During specific operation, a Cas protein used for preparing the ribonucleoprotein complex in this step corresponded to the Cas9 used for the cleavage verification of the sgRNA in step 4.

A reaction is performed at a constant temperature 37° C. for 20 min; and then the reaction is performed at 95° C. for 10 min and then terminated.

### Digestion:

First, 1 μl of RNase A (10 mg/ml) is added and stood at a room temperature for 10 min. Then, 1 μl of proteinase K (20 mg/ml) is added, and a constant temperature 56° C. is maintained for 30 min. Finally, a constant temperature 95° C. is maintained for 10 min.

Constituents of a qPCR system are shown in Table 10.

TABLE 10
qPCR System

| | Final Concentration | |
|---|---|---|
| qPCR F primer | 1 | μm |
| qPCR R primer | 1 | μm |
| dNTP | 0.45 | mM |
| DNA polymerase | 30 | ng/μl |

### Reaction Procedure:

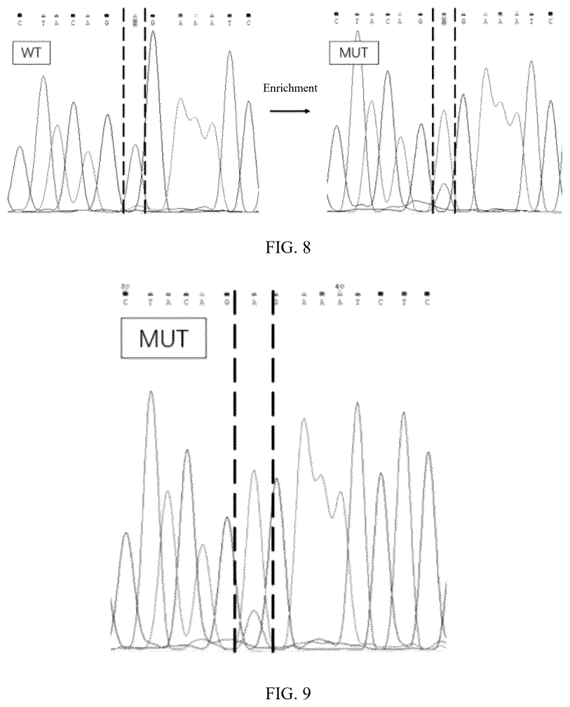

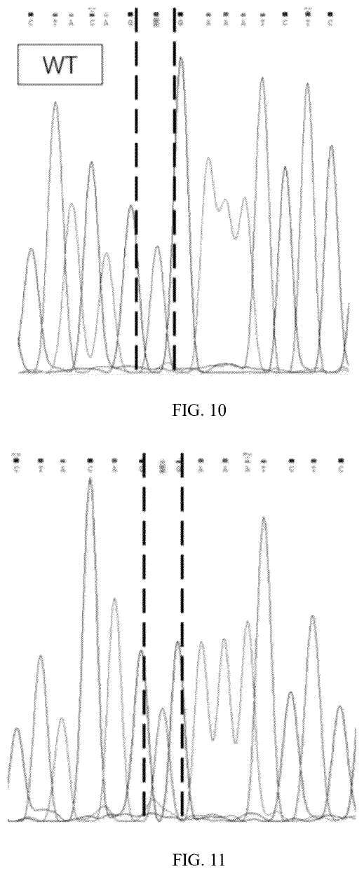

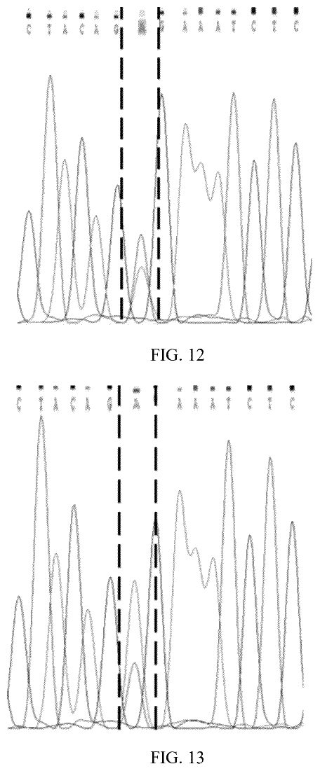

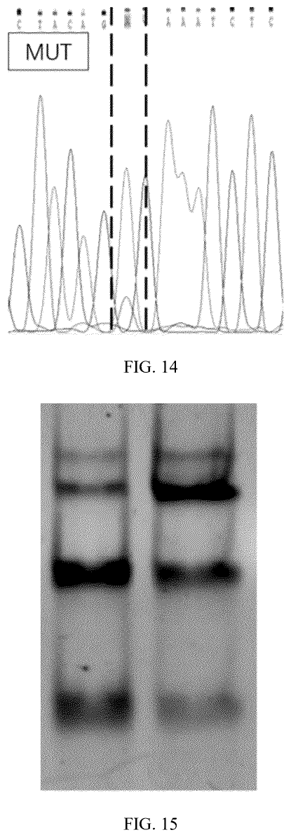

Pre-denaturation: 95° C., 5 min;
45 cycles;
95° C., 10 s;
60° C., 30 s; and
after amplification, sanger sequencing is performed on amplified products, and sequencing results are shown in FIG. 5. To avoid redundancy, only enrichment results of samples having an MAF=0.1% are shown. As can be seen in FIGS. 8-14, wild-type alleles are increased significantly after enrichment. Moreover, by comparing FIGS. 10-14, it can be seen that the enrichment effect is improved significantly with an increase of time of treatment with the Cas protein. It indicates that the enrichment method of the present disclosure can effectively enrich the mutant alleles.

### Example 3

### Enrichment and Detection of EGFR Gene T790M Mutations

### 1, Primers are Designed.

A human EGFR gene sequence is acquired from the NCBI database, and the primers shown as follows are designed according to the sequence:

pre-amplification F primer:
(SEQ ID NO: 23)
TACGTGATGGCCAGCGTGGA
pre-amplification R primer:
(SEQ ID NO: 24)
ACTGGGAGCCAATATTGT;
qPCR F primer:
(SEQ ID NO: 25)
TGATGGCCAGCGTGGACAA;
and
qPCR R primer:
(SEQ ID NO: 26)
TTGTGTTCCCGGACATAGTC;.

2, an sgRNA is Designed.

To be specific, sgRNA sequences having lengths of 17 nt-20 nt and capable of completely matching a wild-type allele sequence are designed as shown in SEQ ID NOS: 27-28:

guide 1:
(SEQ ID NO: 27)
GGCAGCCGAAGGGCAUGAGC.
guide 2:
(SEO ID NO: 28)
GGCAGCCGAAGAGCAUGAGC.

### 3, Genes of Interest are Acquired.

Gene short-chain sequences used as markers in the example are shown as follows:

T790M-WT-S:
(SEQ ID NO: 29)
GACATAGTCCAGGAGGCAGCCGAAGGGCATGAGCTGCGTGATGAG
CTGCACGGTGGAGGTGAGGCAGATGCCCAGCAGGC;
T790M-WT-AS:
(SEQ ID NO: 30)
GCCTGCTGGGCATCTGCCTCACCTCCACCGTGCAGCTCATCACGC
AGCTCATGCCCTTCGGCTGCCTCCTGGACTATGTC;
T790M-MUT-S:
(SEQ ID NO: 31)
GACATAGTCCAGGAGGCAGCCGAAGGGCATGAGCTGCATGATGAG
CTGCACGGTGGAGGTGAGGCAGATGCCCAGCAGGC;
T790M-MUT-AS:
(SEQ ID NO: 32)
GCCTGCTGGGCATCTGCCTCACCTCCACCGTGCAGCTCATCATGC
AGCTCATGCCCTTCGGCTGCCTCCTGGACTATGTC;

the above sequences are synthesized by Sangon Biotech (Shanghai) Co., Ltd.

A genomic DNA is acquired

An EGFR T790M wild-type allele is sourced from a B-CPAP cell line purchased from Shanghai Zhongqiaoxinzhou Biotech Co., Ltd. The genomic DNA is extracted through a QIAGEN DNeasy Blood & Tissue Kit. EGFR T790M mutant allele sequence standard products are purchased from GeneCopoeia. The genomic DNA and the mutant allele sequence standard products are prepared into templates having MAFs=5%, 1%, 0.5%, 0.1%, and 0.01%.

4, Cleavage Verification of the sgRNA is Performed.

### a) Components of a Ribonucleoprotein Complex Shown in Table 11

TABLE 11
Ribonucleoprotein Complex

| | Final Concentration |
|---|---|
| Cas9 | 2.5 μm |
| sgRNA(guide2) | 2.5 μm |
| HEPES | 1M |

Incubation is performed at a constant temperature 37° C. for 10 min.

### b) Constituents of a Cleavage System Shown in Table 12

TABLE 12
Cleavage System

| | Final Concentration |
|---|---|
| Ribonucleoprotein Complex | 2.5 μm |
| MgOAc | 14 mM |
| Template | 0.25 μm |

Cleavage is performed at a constant temperature 37° C. for 1 h.

### c) Digestion

First, 1 μl of RNase A (10 mg/ml) is added and stood at a room temperature for 10 min. Then, 1 μl of proteinase K (20 mg/ml) is added, and a constant temperature 56° C. is maintained for 30 min. Finally, a constant temperature 95° C. is maintained for 10 min.

### d) Gel Running

Polyacrylamide gel electrophoresis (PAGE) is performed. A loading buffer is added into a cleaved product. A sample is added. Electrophoresis is performed at 150 V for 10 min.

After the electrophoresis is ended, staining with a gelred dye is performed for 15 min; and then a photo is taken (see FIG. 15). The two lanes in FIG. 15 denote a wild type and a mutant type from left to right.

### 5, Mutant Alleles are Enriched and Detected.

### a) Pre-Amplification:

Components of a ribonucleoprotein complex are shown in Table 13.

TABLE 13
Ribonucleoprotein Complex

| | Final Concentration |
|---|---|
| Cas9 | 1.4 μm |
| sgRNA | 1.4 μm |
| HEPES | 1M |

Incubation is performed at a constant temperature 37° C. for 10 min, to obtain a ribonucleoprotein complex.

Constituents of a pre-amplification system are shown in Table 14.

TABLE 14
Pre-amplification System

| | Final Concentration | |
|---|---|---|
| Ribonucleoprotein Complex | 1 | μm |
| Pre-amplification F primer | 0.5 | μm |
| Pre-amplification R primer | 0.5 | μm |
| MgOAc | 14 | mM |
| dNTP | 0.45 | mM |
| DNA polymerase | 30 | ng/μl |
| Recombinase | 900 | ng/μl |
| SSB | 150 | ng/μl |
| Template | 60 | ng |

A reaction is performed at a constant temperature 37° C. for 20 min; and then the reaction is performed at 95° C. for 10 min and then terminated.

### Digestion:

First, 1 μl of RNase A (10 mg/ml) is added and stood at a room temperature for 10 min. Then, 1 μl of proteinase K (20 mg/ml) is added, and a constant temperature 56° C. is maintained for 30 min. Finally, a constant temperature 95° C. is maintained for 10 min.

Constituents of a qPCR system are shown in Table 15.

TABLE 15
qPCR System

| | Final Concentration | |
|---|---|---|
| qPCR F primer | 1 | μm |
| qPCR R primer | 1 | μm |
| dNTP | 0.45 | mM |
| DNA polymerase | 30 | ng/μl |

### Reaction Procedure:

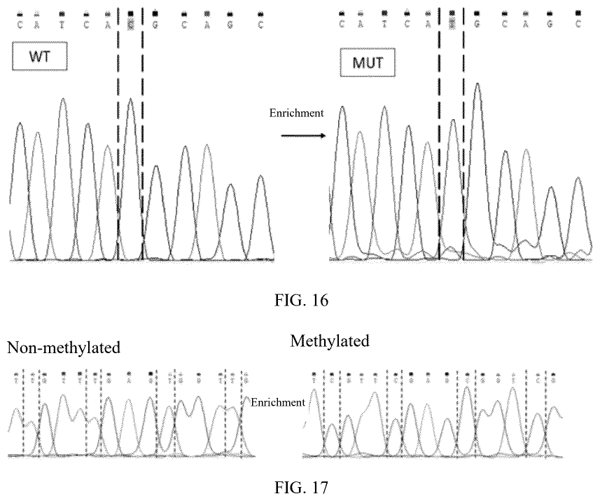

Pre-denaturation: 95° C., 5 min;
45 cycles;
95° C., 10 s;
60° C., 30 s; and
after amplification, sanger sequencing is performed on amplified products, and sequencing results are shown in FIG. 16. To avoid redundancy, only enrichment results of samples having an MAF=5% are shown. As can be seen in FIG. 16, wild-type alleles are increased significantly after enrichment. It indicates that the enrichment method of the present disclosure can effectively enrich the mutant alleles.

### Example 4

### Enrichment and Detection of PCDH10 Gene Methylation

### 1, Primers are Designed.

A human protocadherin 10 (PCDH10) gene sequence is acquired from the NCBI database, and the primers shown as follows are designed according to the sequence:

pre-amplification F primer:
(SEQ ID NO: 33)
TCGTTAAATAGATACGTTACGC;
pre-amplification R primer:
(SEQ ID NO: 34)
TAAAAACTAAAAACTTTCCGCG;
qPCR F primer:
(SEQ ID NO: 35)
TCGTTAAATAGATACGTTACGC;
and
qPCR R primer:
(SEQ ID NO: 36)
CAACGCCTCGAAACCTACG

2, an sgRNA is designed.

An sgRNA sequence having a length of 17 nt-20 nt and capable of completely matching a wild-type allele sequence is designed as shown in SEQ ID NO: 37:
guide: AAUUUUUGUUUGAGUGGUUG (SEQ ID NO: 37);

### 3, a Genomic DNA is Acquired.

A549, a PCDH10 non-methylated cell line, is purchased from Shanghai Zhongqiaoxinzhou Biotech Co., Ltd. The genomic DNA is extracted through a QIAGEN DNeasy Blood & Tissue Kit. The methylated human control is purchased from Promega. Templates with methylation ratios of MAFs=5%, 1%, 0.5%, 0.1%, and 0.01% are prepared.

### 4, Methylated DNAs are Enriched and Detected.

### a) Pre-Amplification:

Components of a ribonucleoprotein complex are shown in Table 16.

TABLE 16
Components of Ribonucleoprotein Complex

| | Final Concentration |
|---|---|
| Cas9 | 1 μm |
| sgRNA | 1 μm |
| HEPES | 1M |

Incubation is performed at a constant temperature 37° C. for 10 min, to obtain a ribonucleoprotein complex.

Constituents of a pre-amplification system are shown in Table 17.

TABLE 17
Pre-amplification System

| | Final Concentration | |
|---|---|---|
| Ribonucleoprotein Complex | 1 | μm |
| Pre-amplification F primer | 0.5 | μm |
| Pre-amplification R primer | 0.5 | μm |
| MgOAc | 14 | mM |
| dNTP | 0.45 | mM |
| DNA polymerase | 30 | ng/μl |
| Recombinase | 900 | ng/μl |
| SSB | 150 | ng/μl |
| Template | 60 | ng |

A reaction is performed at a constant temperature 37° C. for 20 min; and then the reaction is performed at 95° C. for 10 min and then terminated.

### Digestion:

First, 1 μl of RNase A (10 mg/ml) is added and stood at a room temperature for 10 min. Then, 1 μl of proteinase K (20 mg/ml) is added, and a constant temperature 56° C. is maintained for 30 min. Finally, a constant temperature 95° C. is maintained for 10 min.

Constituents of a qPCR system are shown in Table 18.

TABLE 18
qPCR System

| | Final Concentration | |
|---|---|---|
| qPCR F primer | 1 | μm |
| qPCR R primer | 1 | μm |
| dNTP | 0.45 | mM |
| DNA polymerase | 30 | ng/μl |

### Reaction Procedure:

Pre-denaturation: 95° C., 5 min;
30 cycles;
95° C., 10 s;
60° C., 30 s; and
72° C., 30 s.

After amplification, sanger sequencing is performed on amplified products, and sequencing results are shown in FIG. 17. To avoid redundancy, only enrichment results of samples having an MAF=0.1% are shown. As can be seen in FIG. 17, the methylated DNAs are increased significantly after enrichment. It indicates that the enrichment method of the present disclosure can effectively enrich the methylated DNAs.

### Example 5

### Enrichment of NRAS Gene Q61R Mutations

### 1, Primers are Designed.

A gene sequence of an exon 61 of a human NRAS gene is acquired from the NCBI database, and the primers shown as follows are designed according to the sequence:

pre-amplification F primer:
(SEQ ID NO: 38)
CCCCCAGGATTCTTACAGAAAACAAGTGGT;
pre-amplification R primer:
(SEQ ID NO: 39)
GCAAATACACAGAGGAAGCCTTCGCCTGTCCTC;
qPCR F primer:
(SEQ ID NO: 40)
CAAGTGGTTATAGATGGTGA;
and
qPCR R primer:
(SEQ ID NO: 41)
CGCCTGTCCTCATGTATTGG

2, an sgRNA is Designed.

A crRNA sequence having a length of 20 nt and capable of completely matching a wild-type allele sequence is designed as shown in SEQ ID NO: 42:

(SEQ ID NO: 42)
UCCAGCUGUAUCCAGUAUGU;

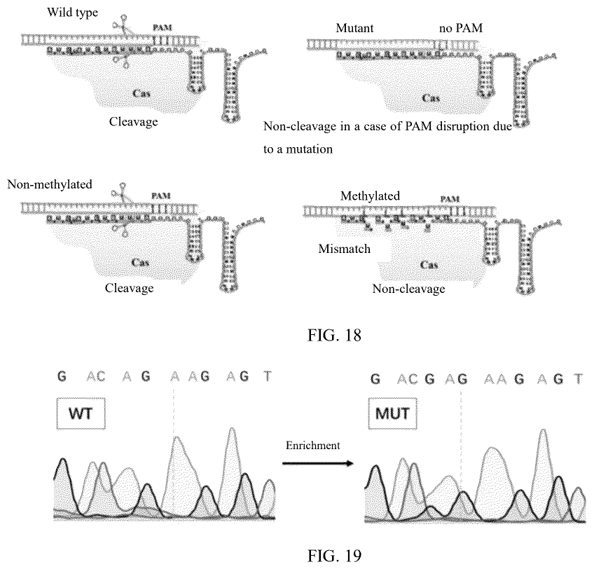

The crRNA is configured to guide a as protein to in with a wild-type allele and specifically cleave the wild-type allele rather than a mutant allele. The principle is as shown in FIG. 18.

### 3, Genes of Interest are Acquired.

An NRAS Q61R wild-type allele is sourced from an IOSE80 cell line, and a mutant allele NRAS Q61R is sourced from an SK-MEL-2 cell line. The cell lines are purchased from Shanghai Zhongqiaoxinzhou Biotech Co., Ltd. Genomic DNAs of two cell lines are extracted through a QIAGEN DNeasy Blood & Tissue Kit according to the instructions. Two genomic DNAs are prepared into templates with mutation ratios of MAFs=10%, 5%, 2.5%, 1%, 0.5%, 0.1%, 0.05%, 0.03%, and 0.01% for enrichment and detection of mutations.

### 4, Mutant Alleles are Enriched and Detected.

### a) Pre-Amplification:

Components of a ribonucleoprotein complex are shown in Table 19.

TABLE 19
Components of Ribonucleoprotein Complex

| | Final Concentration |
|---|---|
| Cas12 | 10 μm |
| crRNA | 10 μm |

A Cas protein herein is FnCas12a. Incubation is performed at a constant temperature 37° C. for 10 min, to obtain the ribonucleoprotein complex.

Constituents of a pre-amplification system are shown in Table 20.

TABLE 20
Pre-amplification System

| | Final Concentration | |
|---|---|---|
| Ribonucleoprotein Complex | 10 | μm |
| Pre-amplification F primer | 0.48 | μm |
| Pre-amplification R primer | 0.48 | μm |
| MgOAc | 14 | mM |
| dNTP | 0.45 | mM |
| DNA polymerase | 30 | ng/μl |
| Recombinase | 900 | ng/μl |
| SSB | 150 | ng/μl |
| Template | 60 | ng |

A reaction is performed at a constant temperature 37° C. for 20 min; and then the reaction is performed at 95° C. for 10 min and then terminated.

### Digestion:

First, 1 μl of RNase A (10 mg/ml) is added and stood at a room temperature for 10 min. Then, 1 μl of proteinase K (20 mg/ml) is added, and a constant temperature 56° C. is maintained for 30 min. Finally, a constant temperature 95° C. is maintained for 10 min.

Constituents of a qPCR system are shown in Table 21.

TABLE 21
qPCR System

| | Final Concentration | |
|---|---|---|
| qPCR F primer | 1 | μM |
| qPCR R primer | 1 | μM |
| dNTP | 0.45 | mM |
| DNA polymerase | 30 | ng/μl |

### Reaction Procedure:

Pre-denaturation: 95° C., 5 min;
45 cycles;
95° C., 10 s;
60° C., 30 s; and
after amplification, sanger sequencing is performed on amplified products, and sequencing results are shown in FIG. 19. To avoid redundancy, only enrichment results of samples having an MAF=0.05% are shown. As can be seen in FIG. 19, wild-type alleles are increased significantly after enrichment. It indicates that the enrichment method of the present disclosure can effectively enrich the mutant alleles.

### Example 6

### Programmable Enzyme Based Selective Exponential Amplification (PASEA) Automation

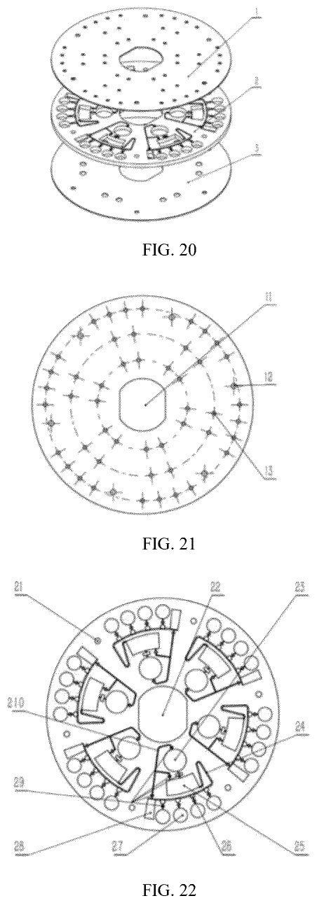

The experimental operations of the enrichment and detection performed in the above examples may be automated through a microfluidic chip. A structure of the microfluidic chip (shown in FIG. 20) is divided into three parts and consists of a top package sheet 1, a bottom package sheet 3, and an intermediate reaction layer 2.

A structure of the top package sheet 1 (shown in FIG. 21) includes a microfluidic chip mounting hole 11; a microfluidic chip package positioning hole 12; and a microfluidic chip sample feeding hole 13.

A structure of the intermediate reaction layer 2 (shown in FIG. 22) includes a microfluidic chip package positioning hole 21; a microfluidic chip mounting hole 22; a pre-amplification reaction chamber 23; a siphon valve 24; a digestion reaction chamber 25; a pre-distribution chamber 26; a polymerase chain reaction (PCR) chamber 27; a waste liquid chamber 28; a capillary valve 29; and a gas passage 210.

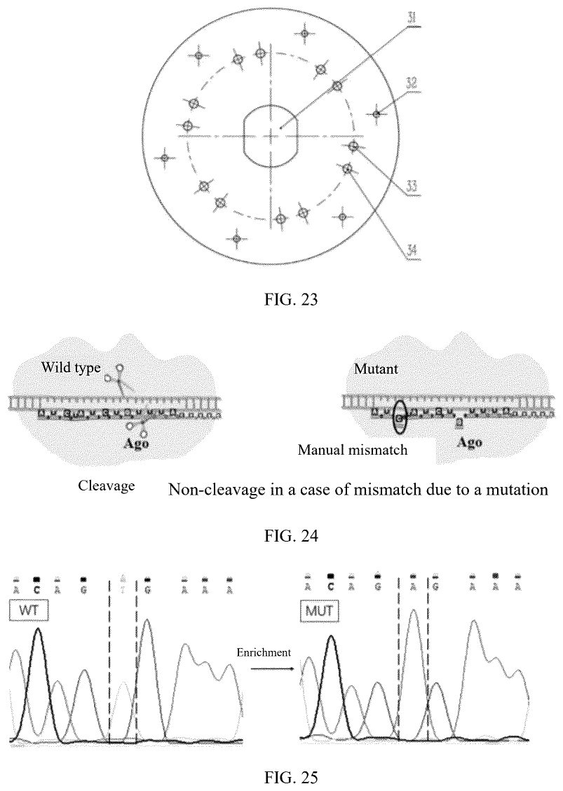

A structure of the bottom package sheet 3 (shown in FIG. 23) includes a microfluidic chip mounting hole 31; a microfluidic chip package positioning hole 32; an RNase A adding chamber 33; and a proteinase K adding chamber 34.

Specific operation steps are as follows:
1) adding a sample: a pre-amplification system is added into the pre-amplification reaction chamber; 5 μl of RNase A (10 mg/ml) is added into the RNase A adding chamber; 5 μl of proteinase K (20 mg/ml) is added into the proteinase K adding chamber; and a qPCR system is added into the PCR chamber.
2) Sealing: a sample feeding opening is sealed with a suitable sealing aluminum foil pressure sensitive film.
3) Loading and experimenting: a sealed microfluidic chip is loaded on a centrifugal microfluidic platform.

The flow is set as follows:
a: a reaction is performed at a constant temperature 37° C. for 20 min (pre-amplification);
b: a rotation speed 3000 rpm is continued for 1 min (a pre-amplification solution is centrifugated into a digestion chamber during rotation, and the siphon valve is opened when the rotation is stopped);
c: a resulting solution is maintained for 10 min without heating (RNase A digestion);
d: a constant temperature 56° C. is maintained for 30 min (proteinase K digestion);
e: a rotation speed 1000 rpm is continued for 1 min (pre-distribution);
f: a rotation speed 3000 rpm is continued for 2 min (a quantitative digested pre-amplification solution is centrifugated into the PCR chamber); and
g: 95° C. is maintained for 5 min; and 45 cycles are performed at 95° C. for 10 s and 60° C. for 30 s (a PCR reaction) separately.

### Example 7

Argonaute (Ago) protein based enrichment and detection of BRAF V600E mutation

### 1, Primers are Designed.

A human BRAF gene sequence is acquired from the NCBI database, and the primers shown as follows are designed according to the sequence:

pre-amplification F primer:
(SEQ ID NO: 19)
CTACACCTCAGATATATTTC;
pre-amplification R primer:
(SEQ ID NO: 20)
TGGATCCAGACAACTGT;
qPCR F primer:
(SEQ ID NO: 21)
CCTCAGATATATTTCTTCATGA;
and
qPCR R primer:
(SEQ ID NO: 22)
TGTTCAAACTGATGGGAC

### 2, a Guide DNA is Designed.

To be specific, guide DNA sequences having lengths of 16 nt-20 nt and capable of completely matching a wild-type allele sequence are designed as shown in SEQ ID NOS: 43-44:

forward guide:
(SEQ ID NO: 43)
p-TAGATTTCACTGTAGC-3′;
and
reverse guide:
(SEQ ID NO: 44)
p-TTCTAGCTACAGTGAA-3′

The guide DNA is configured to guide an Ago protein to bind with a wild-type allele and specifically cleave the wild-type allele rather than a mutant allele. The principle is as shown in FIG. 24.

### 3, Genomic DNAs are Acquired.

A BRAF V600E mutant allele and a BRAF V600E wild-type allele are sourced from a B-CPAP cell line and an HCC827 cell line (purchased from Shanghai Zhongqiaoxinzhou Biotech Co., Ltd) respectively. Genomic DNAs of two cell lines are extracted through a QIAGEN DNeasy Blood & Tissue Kit according to the instructions. Two DNAs are prepared into templates having MAFs=5%, 1%, 0.5%, 0.1%, and 0.01% for enrichment and detection of mutations.

### 5, Mutant Alleles are Enriched and Detected.

### a) Pre-Amplification:

Components of an Ago-guide DNA complex are shown in Table 22.

TABLE 22
Components of Ago-guide DNA complex

| | Final Concentration |
|---|---|
| Ago | 1 μm |
| guide DNA | 10 μm |
| HEPES | 1M |

Incubation is performed at a constant temperature 37° C. for 10 min, to obtain a ribonucleoprotein complex.

Constituents of a pre-amplification system are shown in Table 23.

TABLE 23
Pre-amplification System

| | Final Concentration | |
|---|---|---|
| Ribonucleoprotein Complex | 1 | μm |
| Pre-amplification F primer | 0.5 | μm |
| Pre-amplification R primer | 0.5 | μm |
| MgOAc | 14 | mM |
| dNTP | 0.45 | mM |
| DNA polymerase | 30 | ng/μl |
| Recombinase | 900 | ng/μl |
| SSB | 150 | ng/μl |
| Template | 60 | ng |

The Ago herein includes one of CbAgo, TtAgo, KmAgo, or KpAgo.

A reaction is performed at a constant temperature 37° C. for 20 min; and then the reaction is performed at 95° C. for 10 min and then terminated.

### Digestion:

First, 1 μl of RNase A (10 mg/ml) is added and stood at a room temperature for 10 min. Then, 1 μl of proteinase K (20 mg/ml) is added, and a constant temperature 56° C. is maintained for 30 min. Finally, a constant temperature 95° C. is maintained for 10 min.

Constituents of a qPCR system are shown in Table 24.

TABLE 24
qPCR System

| | Final Concentration | |
|---|---|---|
| qPCR F primer | 1 | μM |
| qPCR R primer | 1 | μM |
| dNTP | 0.45 | mM |
| DNA polymerase | 30 | ng/μl |

### Reaction Procedure:

Pre-denaturation: 95° C., 5 min;
45 cycles;
95° C., 10 s;
60° C., 30 s; and
after amplification, sanger sequencing is performed on amplified products, and sequencing results are shown in FIG. 25. To avoid redundancy, only enrichment results of samples having an MAF=0.1% are shown. As can be seen in FIG. 25, wild-type alleles are increased significantly after enrichment. It indicates that the enrichment method of the present disclosure can effectively enrich the mutant alleles base on specific cleavage of the Ago protein.

The conventional operations in the operation steps of the present disclosure are known to those skilled in the art and are not repeated herein.

The technical solution of the present disclosure is described in detail with reference to the above examples. It should be understood that what are described above are merely the specific examples of the present disclosure, and are not intended to limit the present disclosure. Any modifications, supplements, equivalent replacements, etc. made within the scope of the principles of the present disclosure should fall within the scope of protection of the present disclosure.

What is claimed is:

1. A ribonucleoprotein complex, comprising a first component and a second component; wherein
the first component comprises a nucleic acid region capable of complementarily matching a target nucleic acid and 0 bp-4 bp mismatched fragments; or the nucleic acid region capable of complementarily matching the target nucleic acid, wherein an adjacent protospacer adjacent motif (PAM) comprises a mutation-susceptible site;
the second component is capable of binding with the target nucleic acid and breaking a strand of the target nucleic acid;
the first component is selected from a trans-activating clustered regularly interspaced short palindromic repeats ribonucleic acid (tracrRNA), and/or a clustered regularly interspaced short palindromic repeats ribonucleic acid (crRNA), and/or a single guide RNA (sgRNA), and/or a derivative of the tracrRNA, and/or a derivative of the crRNA, and/or a derivative of the sgRNA, and/or a guide deoxyribonucleic acid (DNA), and/or a guide RNA; the second component comprises a clustered regularly interspaced short palindromic repeats (CRISPR)-associated (Cas) protein, and/or a derivative of the Cas protein, and/or an argonaute (Ago) protein, and/or a derivative of the Ago protein; and the first component and the second component are capable of binding with each other.

2. The ribonucleoprotein complex according to claim 1, wherein a length of a spacer sequence of the sgRNA, the guide DNA, or the guide RNA of the first component is 16 nt-22 nt.

3. The ribonucleoprotein complex according to claim 1, wherein the second component comprises at least one of: Cas12a, SacCas9, CjCas9, SpCas9, NmCas9, Sp-Cas9 HF1, evoCas9, HypaCas9, HiFi Cas9, Sniper-Cas9, xCas9, eSpCas9 1.1, SuperFi Cas9, SaCas9, SaCas9-IF, efSaCas9, ScCas9, Cas9-Sc++, Cas9 HiFi-Sc++, SpaCas9, SpaCas9-IF, FnCas9, AnaCas9, SpyCas9, FnCas12a, LbCas12a, AsCas12a, CbAgo, TtAgo, PfAgo, KmAgo, or KpAgo.

4. A reaction system for enriching mutant genes of a programmable enzyme and/or enriching methylated DNAs, comprising the ribonucleoprotein complex according to claim 1.

5. A method for using the reaction system according to claim 4, for at least one of the following purposes:
(1) target-binding a target nucleic acid fragment;
(2) target-cleaving the target nucleic acid fragment;
(3) enriching the mutant genes;
(4) detecting the mutant genes;
(5) enriching the methylated DNAs; or
(6) detecting the methylated DNAs.

6. The method according to claim 5, wherein the enriching comprises automated enriching; and the detecting comprises automated detecting,
wherein the automated enriching comprises enriching through a microfluidic chip; and the automated detecting comprises detecting through the microfluidic chip.

7. (canceled)

8. The method according to claim 5, wherein the mutant genes comprise a cancer-related gene;
wherein the cancer-related gene comprises at least one of:
EGFR, BRAF, PIK3CA, TP53, LRP1B, APC, CYP1A1, NP01, EPHX1, KRAS, BRCA1, BRCA2, MET, MLH1, MSH2, MSH3, MSH6, PALB2, BMPR1A, SMAD4, STK11, PTEN, AXIN2, BLM, BUB1B, CDH1, CEP57, CHEK2, ENG, EPCAM, FLCN, GALNTI2, GREM1, FAT4, KMT2D, KMT2C, ARID1A, FAT1, PTEN, ATM, ZFHX3, CREBBP, GRIN2A, NRAS, or NF1.

9. (canceled)

10. The method according to claim 5, wherein the methylated DNAs comprise a cancer-related methylated gene and/or a promoter of the cancer-related methylated gene.

11. The method according to claim 10, wherein the cancer-related methylated gene comprises at least one of:
PCDH-10, BRCA1, RASSF1A, ESR1, APC, p14ARF, p161NK4a, DAPK, CDH1, RUNX3, TFPI2, SFRP5, HIC1, PAX5, PGR, THBS1, ESR, COL23A1, C2CD4D, WNT6, OPCML, ZNF154, RARb2, ATM, MGMT, GSTP1, MIR129-2, LINC01158, CCDC181, PRKCB, TBR1, ZNF781, MARCH11, VWC2, SLC9A3, HOXA7, Septin9, IKZF1, BCAT1, hMLH1, WIF1, CDKN2A, SHOX2, 3OST2, ASSF1A, RARb, PITX2, NID2, NEUROG2, or HOXA1;
the promoter comprises at least one of promoters of following genes:
PCDH-10, BRCA1, RASSF1A, ESR1, APC, p14ARF, p16INK4a, DAPK, CDH1, RUNX3, TFPI2, SFRP5, HIC1, PAX5, PGR, THBS1, ESR, COL23A1, C2CD4D, WNT6, OPCML, ZNF154, RARb2, ATM, MGMT, GSTP1, MIR129-2, LINC01158, CCDC181, PRKCB, TBR1, ZNF781, MARCH11, VWC2, SLC9A3, HOXA7, Septin9, IKZF1, BCAT1, hMLH1, WIF1, CDKN2A, SHOX2, 3OST2, ASSF1A, RARb, PITX2, NID2, NEUROG2, or HOXA1.

12. A kit for enriching mutant genes of a programmable enzyme, comprising the ribonucleoprotein complex according to claim 1.

13. The kit according to claim 12, further comprising a constant-temperature cleavage-amplification reaction system, wherein the constant-temperature cleavage-amplification reaction system comprises a primer, a gene of interest, an enzyme, and an auxochrome.

14. A method for enriching mutant genes through the kit according to claim 12, comprising:
collecting a sample;
preparing the ribonucleoprotein complex;
preparing the constant-temperature cleavage-amplification reaction system; and
performing enrichment analysis.

15. An sgRNA, comprising a specific recognition nucleotide fragment and a mismatched nucleotide fragment, wherein a length of the mismatched nucleotide fragment is 0 bp-4 bp.

16. The sgRNA according to claim 15, wherein the sgRNA comprises sequences as follows:
SEQ ID NO[[.]]_ 1: UCUUAAUUCCUUGAUAGCGA;
SEQ ID NO[[.]]_ 2: UAGCUACAGUGAACUCUCGA;
SEQ ID NO[[.]]: 3: UAGCUACAGUGAAAUCACGA;
SEQ ID NO[[.]]: 4: GCUACAGUGAACUCUCGA;
SEQ ID NO[[.]]: 5: GUCUAGCUACAGUGAAA;
SEQ ID NO[[.]]: 6: GUCUAGCUGCAGUGAAA;
SEQ ID NO[[.]]: 27: GGCAGCCGAAGGGCAUGAGC;
SEQ ID NO[[.]]: 28: GGCAGCCGAAGAGCAUGAGC;
SEQ ID NO[[.]]: 37: AAUUUUUGUUUGAGUGGUUG;
SEQ ID NO[[.]]: 42: UCCAGCUGUAUCCAGUAUGU.

17. A method for using the sgRNA according to claim 16, for at least one of the following purposes:
(1) enriching a gene mutation;
(2) detecting the gene mutation;
(3) enriching methylated DNAs; or
(4) detecting the methylated DNAs.

18-20. (canceled)

21. A method for using the Cas protein of the ribonucleoprotein complex according to claim 1, for at least one of the following purposes:
(1) enriching a gene mutation;
(2) detecting the gene mutation;
(3) enriching methylated DNAs; or
(4) detecting the methylated DNAs;
wherein the Cas protein comprises at least one of: Cas12a, SacCas9, CjCas9, SpCas9, NmCas9, Sp-Cas9 HF1, evoCas9, HypaCas9, HiFi Cas9, Sniper-Cas9, xCas9, eSpCas9 1.1, SuperFi Cas9, SaCas9, SaCas9-IF, efSaCas9, ScCas9, Cas9-Sc++, Cas9 HiFi-Sc++, SpaCas9, SpaCas9-IF, FnCas9, AnaCas9, SpyCas9, FnCas12a, LbCas12a, AsCas12a, CbAgo, TtAgo, PfAgo, KmAgo, or KpAgo.

22. A method for using an Ago protein, for at least one of the following purposes:
(1) enriching a gene mutation;
(2) detecting the gene mutation;
(3) enriching methylated DNAs; or
(4) detecting the methylated DNAs;
wherein the Ago protein comprises at least one of: CbAgo, TtAgo, PfAgo, KmAgo, or KpAgo.

23. The ribonucleoprotein complex according to claim 1, wherein the guide DNA or the guide RNA comprises a specific recognition nucleotide fragment and a mismatched nucleotide fragment, wherein a length of the mismatched nucleotide fragment is 0 bp-4 bp.

24. The ribonucleoprotein complex according to claim 23, wherein the guide DNA comprises sequences as follows:
SEQ ID NO: 43:
forward guide:
p-TAGATTTCACTGTAGC-3′;
and
SEQ ID NO: 44:
reverse guide:
p-TTCTAGCTACAGTGAA-3′.

25. The ribonucleoprotein complex according to claim 23, wherein use of the guide DNA or the guide RNA comprises at least one of:
(1) enriching a gene mutation;
(2) detecting the gene mutation;
(3) enriching methylated DNAs; or
(4) detecting the methylated DNAs.


SEQUENCE LISTING (44 sequences)

SEQ ID NO: 1 - RNA, 20 nt, synthetic construct
  source 1..20: mol_type = other RNA
tcttaattccttgatagcga

SEQ ID NO: 2 - RNA, 20 nt, synthetic construct
  source 1..20: mol_type = other RNA
tagctacagtgaactctcga

SEQ ID NO: 3 - RNA, 20 nt, synthetic construct
  source 1..20: mol_type = other RNA
tagctacagtgaaatcacga

SEQ ID NO: 4 - RNA, 18 nt, synthetic construct
  source 1..18: mol_type = other RNA
gctacagtgaactctcga

SEQ ID NO: 5 - RNA, 17 nt, synthetic construct
  source 1..17: mol_type = other RNA
gtctagctacagtgaaa

SEQ ID NO: 6 - RNA, 17 nt, synthetic construct
  source 1..17: mol_type = other RNA
gtctagctgcagtgaaa

SEQ ID NO: 7 - DNA, 100 bp, synthetic construct
  source 1..100: mol_type = other DNA
ctctggatcccagaaggtgagaaagttaaaattcccgtcgctatcaaggaattaagagaagcaacatctccgaaagccaacaaggaaatcctcgatgtga

SEQ ID NO: 8 - DNA, 100 bp, synthetic construct
  source 1..100: mol_type = other DNA
tcacatcgaggatttccttgttggctttcggagatgttgcttctcttaattccttgatagcgacgggaattttaactttctcaccttctgggatccagag

SEQ ID NO: 9 - DNA, 100 bp, synthetic construct
  source 1..100: mol_type = other DNA
ctctggatcccagaaggtgagaaagttaaaattcccgtcgctatcaaaacatctccgaaagccaacaaggaaatcctcgatgtgagtttctgctttgctg

SEQ ID NO: 10 - DNA, 100 bp, synthetic construct
  source 1..100: mol_type = other DNA
cagcaaagcagaaactcacatcgaggatttccttgttggctttcggagatgttttgatagcgacgggaattttaactttctcaccttctgggatccagag

SEQ ID NO: 11 - DNA, 100 bp, synthetic construct
  source 1..100: mol_type = other DNA
ctctggatcccagaaggtgagaaagttaaaattcccgtcgctatcaagacatctccgaaagccaacaaggaaatcctcgatgtgagtttctgctttgctg

SEQ ID NO: 12 - DNA, 100 bp, synthetic construct
  source 1..100: mol_type = other DNA
cagcaaagcagaaactcacatcgaggatttccttgttggctttcggagatgtcttgatagcgacgggaattttaactttctcaccttctgggatccagag

SEQ ID NO: 13 - DNA, 100 bp, synthetic construct
  source 1..100: mol_type = other DNA
tctggatcccagaaggtgagaaagttaaaattcccgtcgctatcaaggaatcgaaagccaacaaggaaatcctcgatgtgagtttctgctttgctgtgtg

SEQ ID NO: 14 - DNA, 100 bp, synthetic construct
  source 1..100: mol_type = other DNA
cacacagcaaagcagaaactcacatcgaggatttccttgttggctttcgattccttgatagcgacgggaattttaactttctcaccttctgggatccaga

SEQ ID NO: 15 - DNA, 20 bp, synthetic construct
  source 1..20: mol_type = other DNA
gcatgtggcaccatctcaca

SEQ ID NO: 16 - DNA, 20 bp, synthetic construct
  source 1..20: mol_type = other DNA
agagcagctgccagacatga

SEQ ID NO: 17 - DNA, 25 bp, synthetic construct
  source 1..25: mol_type = other DNA
tgtcatagggactctggatcccaga

SEQ ID NO: 18 - DNA, 29 bp, synthetic construct
  source 1..29: mol_type = other DNA
gcagaaactcacatcgaggatttccttgt

SEQ ID NO: 19 - DNA, 20 bp, synthetic construct
  source 1..20: mol_type = other DNA
ctacacctcagatatatttc

SEQ ID NO: 20 - DNA, 17 bp, synthetic construct
  source 1..17: mol_type = other DNA
tggatccagacaactgt

SEQ ID NO: 21 - DNA, 22 bp, synthetic construct
  source 1..22: mol_type = other DNA
cctcagatatatttcttcatga

SEQ ID NO: 22 - DNA, 18 bp, synthetic construct
  source 1..18: mol_type = other DNA
tgttcaaactgatgggac

SEQ ID NO: 23 - DNA, 20 bp, synthetic construct
  source 1..20: mol_type = other DNA
tacgtgatggccagcgtgga

SEQ ID NO: 24 - DNA, 18 bp, synthetic construct
  source 1..18: mol_type = other DNA
actgggagccaatattgt

SEQ ID NO: 25 - DNA, 19 bp, synthetic construct
  source 1..19: mol_type = other DNA
tgatggccagcgtggacaa

SEQ ID NO: 26 - DNA, 20 bp, synthetic construct
  source 1..20: mol_type = other DNA
ttgtgttcccggacatagtc

SEQ ID NO: 27 - RNA, 20 nt, synthetic construct
  source 1..20: mol_type = other RNA
ggcagccgaagggcatgagc

SEQ ID NO: 28 - RNA, 20 nt, synthetic construct
  source 1..20: mol_type = other RNA
ggcagccgaagagcatgagc

SEQ ID NO: 29 - DNA, 80 bp, synthetic construct
  source 1..80: mol_type = other DNA
gacatagtccaggaggcagccgaagggcatgagctgcgtgatgagctgcacggtggaggtgaggcagatgcccagcaggc

SEQ ID NO: 30 - DNA, 80 bp, synthetic construct
  source 1..80: mol_type = other DNA
gcctgctgggcatctgcctcacctccaccgtgcagctcatcacgcagctcatgcccttcggctgcctcctggactatgtc

SEQ ID NO: 31 - DNA, 80 bp, synthetic construct
  source 1..80: mol_type = other DNA
gacatagtccaggaggcagccgaagggcatgagctgcatgatgagctgcacggtggaggtgaggcagatgcccagcaggc

SEQ ID NO: 32 - DNA, 80 bp, synthetic construct
  source 1..80: mol_type = other DNA
gcctgctgggcatctgcctcacctccaccgtgcagctcatcatgcagctcatgcccttcggctgcctcctggactatgtc

SEQ ID NO: 33 - DNA, 22 bp, synthetic construct
  source 1..22: mol_type = other DNA
tcgttaaatagatacgttacgc

SEQ ID NO: 34 - DNA, 22 bp, synthetic construct
  source 1..22: mol_type = other DNA
taaaaactaaaaactttccgcg

SEQ ID NO: 35 - DNA, 22 bp, synthetic construct
  source 1..22: mol_type = other DNA
tcgttaaatagatacgttacgc

SEQ ID NO: 36 - DNA, 19 bp, synthetic construct
  source 1..19: mol_type = other DNA
caacgcctcgaaacctacg

SEQ ID NO: 37 - RNA, 20 nt, synthetic construct
  source 1..20: mol_type = other RNA
aatttttgtttgagtggttg

SEQ ID NO: 38 - DNA, 30 bp, synthetic construct
  source 1..30: mol_type = other DNA
cccccaggattcttacagaaaacaagtggt

SEQ ID NO: 39 - DNA, 33 bp, synthetic construct
  source 1..33: mol_type = other DNA
gcaaatacacagaggaagccttcgcctgtcctc

SEQ ID NO: 40 - DNA, 20 bp, synthetic construct
  source 1..20: mol_type = other DNA
caagtggttatagatggtga

SEQ ID NO: 41 - DNA, 20 bp, synthetic construct
  source 1..20: mol_type = other DNA
cgcctgtcctcatgtattgg

SEQ ID NO: 42 - RNA, 20 nt, synthetic construct
  source 1..20: mol_type = other RNA
tccagctgtatccagtatgt

SEQ ID NO: 43 - DNA, 16 bp, synthetic construct
  source 1..16: mol_type = other DNA
tagatttcactgtagc

SEQ ID NO: 44 - DNA, 16 bp, synthetic construct
  source 1..16: mol_type = other DNA
ttctagctacagtgaa